(19) United States
(12) Patent Application Publication
(10) Pub. No.: US 2025/0099776 A1
(43) Pub. Date: Mar. 27, 2025

(54) PHOTOTHERAPEUTIC ILLUMINATION DEVICES WITH MODULAR STRUCTURES

(71) Applicant: KNOW Bio, LLC, Morrisville, NC (US)

(72) Inventors: Thomas Matthew Womble, Wake Forest, NC (US); Sameer Tendulkar, Raleigh, NC (US); P. Joseph DeSena, JR., Raleigh, NC (US); James Michael Lay, Apex, NC (US); F. Neal Hunter, Durham, NC (US)

(21) Appl. No.: 18/472,349

(22) Filed: Sep. 22, 2023

(51) Int. Cl. A61N 5/06

(52) U.S. Cl. CPC A61N 5/0603; A61N 5/0624; A61N 2005/0606; A61N 2005/0626; A61N 2005/063; A61N 2005/0632; A61N 2005/0662; A61N 2005/0664

(57) ABSTRACT

Illumination devices for directing light on tissue to induce one or more biological effects and more particularly phototherapeutic illumination devices with modular structures are disclosed. Modular structures are disclosed that provide the ability to have various changeable attributes for illumination devices based on intended treatment protocols. Modular structures may include tokens, switches, handles, rechargeable power structures, control modules, illumination modules, optics, light guides, and/or light guide positioners, among others.

### FIELD OF THE DISCLOSURE

The present disclosure relates generally to devices for directing light on tissue to induce one or more biological effects and more particularly to phototherapeutic illumination devices with modular structures.

### BACKGROUND

Disease-causing pathogens typically invade tissues of the human body via mucosal surfaces within body cavities, such as mucous membranes or mucosae of the respiratory tract. A number of respiratory diseases and infections, including viral and bacterial, can be attributed to such disease-causing pathogens. Examples include Orthomyxoviridae (e.g., influenza), common colds, coronaviridae (e.g., coronavirus), picornavirus infections, tuberculosis, pneumonia, bronchitis, and sinusitis. Most respiratory tract infections begin when a subject is exposed to pathogen particles, which enter the body through the mouth and nose. For viral infections, cells at the site of infection must be accessible, susceptible, and permissive for the virus, and local host anti-viral defense systems must be absent or initially ineffective. Conventional treatments for infections may involve systemic administration of antimicrobials, such as antibiotics for bacterial infections, that can sometimes lead to drug resistance and gastro-intestinal distress. Other conventional treatment protocols may involve managing and enduring symptoms while waiting for infections to clear, particularly in the case of viral infections.

Upper respiratory tract infections, including the common cold, influenza, and those resulting from exposure to coronaviridae, are widely prevalent infections that continually impact the worldwide population. In some instances, upper respiratory tract infections can progress to cause serious and sometimes fatal diseases that develop in the lower respiratory tract or elsewhere in the body. The art continues to seek improved treatment options for respiratory tract conditions capable of overcoming challenges associated with conventional treatment options.

### SUMMARY

The present disclosure relates generally to devices for directing light on tissue to induce one or more biological effects and more particularly to phototherapeutic illumination devices with modular structures. Modular structures are disclosed that provide the ability to have various changeable attributes for illumination devices based on intended treatment protocols. Modular structures may include tokens, switches, handles, rechargeable power structures, control modules, illumination modules, optics, light guides, and/or light guide positioners, among others.

In one aspect, an illumination device comprises: at least one light source arranged to irradiate light on tissue to induce a biological effect; driver circuitry configured to drive the at least one light source; and a removable token configured to control operation of the at least one light source. In certain embodiments, the removable token is configured for near field communication with the driver circuitry. The illumination device may further comprise a housing that at least partially encloses the at least one light source and the driver circuitry. In certain embodiments, the housing comprises a receptacle configured to receive the removable token. In certain embodiments, the removable token forms a protrusion with a first shape that corresponds with a second shape of a slot of the receptacle. In certain embodiments, the removable token comprises a plug that fits into the receptacle. In certain embodiments, the removable token forms a physical connection within the receptacle for providing transfer of information. In certain embodiments, the removable token is configured to provide near field communication for transfer of information with the driver circuitry. In certain embodiments, the housing comprises a window proximate the at least one light source configured to provide visual indication when the at least one light source is electrically activated. The illumination device may further comprise: a heatsink within the housing, wherein the at least one light source is on the heatsink; and an optical cup peripherally surrounding the at least one light source, wherein a portion of the optical cup is attached to the heatsink.

In certain embodiments, the housing forms a handle and the removable token is attached to a base of the handle. The illumination device may further comprise a carriage within the housing, wherein the removable token is attached to the carriage. The illumination device may further comprise a port at the carriage, the port being configured as one or more of a charging port and a data transfer port. In certain embodiments, the driver circuitry is attached to the carriage.

In certain embodiments, the removable token is configured to slide within the receptacle to lock in place.

The illumination device may further comprise: a light guide attached to the housing and optically coupled to the at least one light source; and a light guide positioner attached to the light guide, wherein the light guide positioner is configured to be positioned within an oral cavity of a user. The illumination device may comprise a user interface display on the housing.

In certain embodiments, the removable token is configured to be prescribed by a health care provider. In certain embodiments, the removable token is configured to control operation of the at least one light source according to a treatment protocol for irradiating light on tissue within a body cavity. In certain embodiments, the removable token forms a switch electrically coupled to the driver circuitry for initiating operation of the at least one light source. The illumination device may further comprise a communication module configured to communication with an external device. In certain embodiments, the removable token is part of an illumination cartridge that includes the at least one light source, the illumination cartridge being removable. In certain embodiments, the removable token is removable from the illumination cartridge. In certain embodiments, the removable token is part of an illumination head that includes the at least one light source, the illumination head being removable. In certain embodiments, the removable token is removable from the illumination head.

In another aspect, an illumination device comprises: a housing; and an illumination cartridge configured to be releasably attached to the housing, the illumination cartridge comprising at least one light source arranged to irradiate light on tissue to induce a biological effect; and driver circuitry configured to drive the at least one light source. The illumination device may further comprise a light guide attached to the housing and optically coupled to the at least one light source. The illumination device may further comprise a light guide positioner attached to the light guide, wherein the light guide positioner is configured to be positioned within an oral cavity of a user. In certain embodiments, the illumination cartridge further comprises a cartridge window configured to provide visual indication when the at least one light source is electrically activated. In certain embodiments, the cartridge window is positioned to receive a portion of light from the at least one light source. In certain embodiments, the illumination cartridge further comprises an additional light source that is spaced apart from the at least one light source, wherein the cartridge window is positioned to receive a portion of light from the additional light source. In certain embodiments, the cartridge window comprises a button for initiating operation of the at least one light source. In certain embodiments, the illumination cartridge further comprises a heat sink. In certain embodiments, the illumination cartridge further comprises an optical cup peripherally surrounding the at least one light source, wherein a portion of the optical cup is attached to the heatsink. In certain embodiments, the illumination cartridge further comprises a base that is coupled to the optical cup, wherein a portion of the base is connected to the housing. The illumination device may further comprise a user interface display on the housing. In certain embodiments, the driver circuitry is within the illumination cartridge. In certain embodiments, the driver circuitry is within the housing. In certain embodiments, the illumination cartridge comprises a token configured to control operation of the at least one light source. In certain embodiments, the token is removable from the illumination cartridge.

In another aspect, an illumination device comprises: an illumination head comprising at least one light source arranged to irradiate light on tissue to induce a biological effect; driver circuitry configured to drive the at least one light source; and a handle configured for releasable attachment with the illumination head. In certain embodiments, the illumination head comprises an illumination head window configured to provide visual indication when the at least one light source is electrically activated. The illumination head may further comprise an additional light source that is spaced apart from the at least one light source, wherein the illumination head window is positioned to receive a portion of light from the additional light source. In certain embodiments, the illumination head window comprises a button for initiation operation of the at least one light source. In certain embodiments, the illumination head further comprises a heat sink. In certain embodiments, the handle comprises an energy storage device and a port, wherein the port is one or more of a charging port and a data transfer port. In certain embodiments, the port is positioned at a base of the handle. The illumination device may further comprise: a light guide attached to the illumination head and optically coupled to the at least one light source; and a light guide positioner attached to the light guide, wherein the light guide positioner is configured to be positioned within an oral cavity of a user. The illumination device may further comprise a user interface display on the illumination head. In certain embodiments, the driver circuitry is positioned within the illumination head. In certain embodiments, the driver circuitry is positioned within the handle. In certain embodiments, the illumination head comprises a token configured to control operation of the at least one light source. In certain embodiments, the token is removable from the illumination head.

In another aspect, an illumination device comprises: a housing; at least one light source within the housing; a mouthpiece attached to the housing and configured to engage with one or more surfaces of an oral cavity; and a window formed in the housing and spaced away from the mouthpiece, the window configured to provide a visual indication when the at least one light source is electrically activated. In certain embodiments, the window is positioned to receive a portion of light from the at least one light source. The illumination device may further comprise an additional light source that is spaced apart from the at least one light source, wherein the window is positioned to receive a portion of light from the additional light source. The illumination device may further comprise a light guide attached to the housing and optically coupled to the at least one light source, wherein the mouthpiece is arranged about a periphery of the light guide. The illumination device may further comprise a removable token configured to control operation of the at least one light source. In certain embodiments, the housing comprises a receptacle configured to receive the removable token.

In another aspect, an illumination device comprises: a housing; a heatsink within the housing; at least one light source on the heatsink, the at least one light source arranged to irradiate light on tissue to induce a biological effect; and an optical cup peripherally surrounding the at least one light source and a portion of the optical cup being attached to the heatsink. In certain embodiments, the heatsink forms a protruding lip and the portion of the optical cup is attached to the protruding lip. In certain embodiments, the heatsink comprises a mounting surface for the at least one light source and the protruding lip extends in a direction parallel to the mounting surface. The illumination device may further comprise a lens within the housing, wherein the optical cup is between the lens and the at least one light source. In certain embodiments, the housing comprises a window proximate the at least one light source, and the optical cup comprises an opening that is registered with the window. The illumination device may further comprise a light guide attached to the housing and optically coupled to the at least one light source, wherein the optical cup extends between the heatsink and the light guide. The illumination device may further comprise a removable token configured to control operation of the at least one light source. In certain embodiments, the housing comprises a receptacle configured to receive the removable token.

In another aspect, any of the foregoing aspects individually or together, and/or various separate aspects and features as described herein, may be combined for additional advantage. Any of the various features and elements as disclosed herein may be combined with one or more other disclosed features and elements unless indicated to the contrary herein.

Those skilled in the art will appreciate the scope of the present disclosure and realize additional aspects thereof after reading the following detailed description of the preferred embodiments in association with the accompanying drawing figures.

### BRIEF DESCRIPTION OF THE DRAWING FIGURES

The accompanying drawing figures incorporated in and forming a part of this specification illustrate several aspects of the disclosure, and together with the description serve to explain the principles of the disclosure.

FIG. 1A is front perspective view of an illumination device according to the present disclosure.

FIG. 1B is a back perspective view of the illumination device of FIG. 1A.

FIG. 1C is a perspective view of a token of FIG. 1B from a side that faces a housing when positioned within a receptacle of the illumination device of FIG. 1B.

FIG. 1D is a back perspective view of the illumination device of FIGS. 1A and 1B illustrating removable attachment capabilities of the token.

FIG. 1E is a cross-sectional view of the illumination device of FIGS. 1A and 1B.

FIG. 1F is an exploded view of the illumination device of FIG. 1E.

FIG. 2A is a front perspective view of an illumination device that is similar to the illumination device of FIG. 1A.

FIG. 2B is a back perspective view of the illumination device of FIG. 2A illustrating a different arrangement of the token and receptacle as compared with the illumination device of FIG. 1B.

FIG. 2C is a perspective view of the token of FIG. 2B.

FIG. 2D is a cross-sectional view of the illumination device of FIGS. 2A and 2B.

FIG. 2E is an exploded view of the illumination device of FIG. 2D.

FIG. 3A is a back perspective view of an illumination device that is similar to the illumination device of FIG. 2B except the token is attached to a base of the housing.

FIG. 3B is a back perspective view of the illumination device of FIG. 3A with the token illustrated as detached from the housing.

FIG. 4A is a front perspective view of an illumination device that is similar to the illumination device of FIGS. 3A and 3B with a different attachment structure for the token.

FIG. 4B is a back perspective view of the illumination device of FIG. 4A.

FIG. 4C is a perspective view of the token of FIG. 4A.

FIG. 4D is a perspective view of the illumination device of FIG. 4A with the token illustrated in exploded view relative to the housing.

FIG. 4E is a cross-sectional view of the illumination device of FIGS. 4A to 4D.

FIG. 4F is an exploded view of the illumination device of FIGS. 4A to 4D.

FIG. 5A is a side view of an illumination device that is similar to the illumination device of FIG. 1A except the token also forms a switch for initiating operation of the illumination device.

FIG. 5B is an exploded side view of the illumination device of FIG. 5A.

FIG. 5C is a cross-sectional view of the illumination device of FIG. 5A.

FIG. 5D is front view of the illumination device of FIG. 5C with the housing represented as transparent for illustrative purposes.

FIG. 6A is an exploded side view of an illumination device that is similar to the illumination device of FIGS. 5A to 5D except the illumination device includes an illumination cartridge for attachment with the housing.

FIG. 6B is a front view of the illumination cartridge of FIG. 6A.

FIG. 6C is a front view of the housing of the illumination device of FIG. 6A.

FIG. 6D is a front view of the illumination device of FIG. 6A with the illumination cartridge in place.

FIG. 6E is a side view of the illumination device of FIG. 6A with the illumination cartridge attached in place.

FIG. 6F is a cross-sectional view of the illumination device of FIG. 6E.

FIG. 6G is a cross-sectional view of the illumination cartridge of FIG. 6F.

FIG. 7A is an exploded side view of an illumination device that is similar to the illumination device of FIGS. 6A to 6G for embodiments where the housing of previous embodiments comprises an illumination head and a detachable handle.

FIG. 7B is a front view of the illumination device of FIG. 7A.

FIG. 7C is a side view of the illumination device of FIG. 7A with the handle detached from the illumination head.

FIG. 7D is a cross-sectional view of the illumination device of FIG. 7A.

FIG. 7E is a front view of the illumination device of FIG. 7D with portions of the illumination head and the handle represented as transparent for illustrative purposes.

FIG. 8A is a side view of another embodiment of an illumination device that is similar to the illumination device of FIG. 7C with the handle detached from the illumination head.

FIG. 8B is a side view of the illumination device of FIG. 8A with the handle attached to the illumination head.

FIG. 8C is a back perspective view of the illumination device of FIG. 8A.

FIG. 8D is a front view of the illumination device of FIG. 8C with a portion of the handle shown as transparent for illustrative purposes.

FIG. 8E is a cross-sectional view of the illumination device of FIG. 8A with the addition of a light guide and light guide positioner.

FIG. 9A is a side view of an illumination device that is similar to the illumination device of FIGS. 6A to 6B with a different structure for the illumination cartridge according to further aspects of the present disclosure.

FIG. 9B is a front view of the illumination device of FIG. 9A.

FIG. 9C is a back perspective view of the illumination device of FIG. 9A.

FIG. 9D is a cross-sectional view of the illumination device of FIG. 9A with the addition of the light guide and light guide positioner.

FIG. 9E is a front perspective view of the illumination device of FIG. 9A with the illumination cartridge detached from the housing.

FIG. 9F is an exploded view of the illumination cartridge of FIG. 9A.

FIG. 10 is a schematic view of an exemplary illumination device configured to communicate with one or more local devices, servers, and networks according to embodiments of the present disclosure.

### DETAILED DESCRIPTION

The embodiments set forth below represent the necessary information to enable those skilled in the art to practice the embodiments and illustrate the best mode of practicing the embodiments. Upon reading the following description in light of the accompanying drawing figures, those skilled in the art will understand the concepts of the disclosure and will recognize applications of these concepts not particularly addressed herein. It should be understood that these concepts and applications fall within the scope of the disclosure and the accompanying claims.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of the present disclosure. As used herein, the term “and/or” includes any and all combinations of one or more of the associated listed items.

It will be understood that when an element such as a layer, region, or substrate is referred to as being “on” or extending “onto” another element, it can be directly on or extend directly onto the other element or intervening elements may also be present. In contrast, when an element is referred to as being “directly on” or extending “directly onto” another element, there are no intervening elements present. Likewise, it will be understood that when an element such as a layer, region, or substrate is referred to as being “over” or extending “over” another element, it can be directly over or extend directly over the other element or intervening elements may also be present. In contrast, when an element is referred to as being “directly over” or extending “directly over” another element, there are no intervening elements present. It will also be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present.

Relative terms such as “below” or “above” or “upper” or “lower” or “horizontal” or “vertical” may be used herein to describe a relationship of one element, layer, or region to another element, layer, or region as illustrated in the Figures. It will be understood that these terms and those discussed above are intended to encompass different orientations of the device in addition to the orientation depicted in the Figures.

The terminology used herein is for the purpose of describing particular embodiments only and is not intended to be limiting of the disclosure. As used herein, the singular forms “a,” “an,” and “the” are intended to include the plural forms as well, unless the context clearly indicates otherwise. It will be further understood that the terms “comprises,” “comprising,” “includes,” and/or “including” when used herein specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this disclosure belongs. It will be further understood that terms used herein should be interpreted as having a meaning that is consistent with their meaning in the context of this specification and the relevant art and will not be interpreted in an idealized or overly formal sense unless expressly so defined herein.

Embodiments are described herein with reference to schematic illustrations of embodiments of the disclosure. As such, the actual dimensions of the layers and elements can be different, and variations from the shapes of the illustrations as a result, for example, of manufacturing techniques and/or tolerances, are expected. For example, a region illustrated or described as square or rectangular can have rounded or curved features, and regions shown as straight lines may have some irregularity. Thus, the regions illustrated in the figures are schematic and their shapes are not intended to illustrate the precise shape of a region of a device and are not intended to limit the scope of the disclosure. Additionally, sizes of structures or regions may be exaggerated relative to other structures or regions for illustrative purposes and, thus, are provided to illustrate the general structures of the present subject matter and may or may not be drawn to scale. Common elements between figures may be shown herein with common element numbers and may not be subsequently re-described.

The present disclosure relates generally to devices for directing light on tissue to induce one or more biological effects and more particularly to phototherapeutic illumination devices with modular structures. Modular structures are disclosed that provide the ability to have various changeable attributes for illumination devices based on intended treatment protocols. Modular structures may include tokens, switches, handles, rechargeable power structures, control modules, illumination modules, optics, light guides, and/or light guide positioners, among others.

Aspects of the present disclosure relate to devices and methods for directing light on a mammalian tissue, for example within a body and/or a body cavity of a patient, where the light may include at least one characteristic that exerts or induces at least one biological effect within or on the tissue. Exemplary tissues include those of the upper respiratory tract, including tissues and cavities that are accessible via the oral cavity, such as the nasopharynx and the oropharynx. Biological effects may include at least one of inactivating and inhibiting growth of one or more combinations of microorganisms and pathogens, including but not limited to viruses, bacteria, fungi, and other microbes, among others. Biological effects may also include one or more of upregulating and/or downregulating a local immune response, stimulating enzymatic generation of nitric oxide to increase endogenous stores of nitric oxide, releasing nitric oxide from endogenous stores of nitric oxide, and inducing an anti-inflammatory effect. Wavelengths of light may be selected based on at least one intended biological effect for one or more of the targeted tissues and the targeted microorganisms and/or pathogens. In certain aspects, wavelengths of light may include visible light in any number of wavelength ranges based on the intended biological effect. Further aspects involve directing light on tissue for multiple microorganisms and/or multiple pathogenic biological effects, either with light of a single peak wavelength or a combination of light with more than one peak wavelength. Devices and methods for light treatments are disclosed that provide light doses for inducing biological effects on various targeted pathogens and targeted tissues with increased efficacy and reduced cytotoxicity. Light doses may include various combinations of irradiances, wavelengths, and exposure times, and such light doses may be administered continuously or discontinuously with a number of pulsed exposures.

Exemplary devices and methods relate to treating, preventing, and/or reducing the biological activity of pathogens while they are in one or more areas of the upper respiratory tract and hopefully before they travel to the lungs or elsewhere in the body. Devices and methods as disclosed herein may prevent or reduce infections by reducing microbial load along one or more portions of the upper respiratory tract, decreasing the ability for penetration into cells at the site of infection, and amplifying host defense systems, all of which may reduce or avoid the need for traditional antimicrobial medicines. In further aspects, devices and methods for light irradiation of tissues as disclosed herein may be provided to supplement and/or enhance the effects of traditional antimicrobial medicines.

Embodiments of the present disclosure may administer light at one or more wavelengths as a pre-exposure prophylaxis or a post-exposure prophylaxis in order to target pathogens in or on tissue of the upper respiratory tract and/or amplify host defense systems. Embodiments of the present disclosure may be used to prevent and/or treat respiratory infections and other infectious diseases. For example, in certain embodiments, a hand-held illumination device may administer light at one or more wavelengths as a prophylactic measure to counteract invading viral pathogens and corresponding diseases that may originate in the respiratory tract. In a specific example, light may be administered that reduces viral infectivity and incidence of COVID-19 in individuals who have been infected or believe they may have been exposed to SARS-COV-2 virus. In certain aspects, illumination devices of the present disclosure may be referred to as phototherapeutic and/or phototherapy devices.

The term “phototherapy” or “phototherapeutic” generally relates to the therapeutic use of light for, by way of example, treating and/or preventing microbial infections, including viral infections of the upper respiratory tract. The mechanisms by which certain wavelengths of light are effective can vary, depending on the wavelength that is administered and the targeted microorganisms and/or pathogens. Biological effects, including antimicrobial effects, can be provided over a wide range of wavelengths, including ultraviolet (UV) ranges, visible light ranges, and infrared (IR) ranges, and combinations thereof.

Various wavelengths of visible light may be irradiated on human tissue with little or no impact on tissue viability. In certain embodiments, various wavelengths of visible light may elicit antimicrobial and/or anti-pathogenic behavior in tissue of the respiratory tract, including any of the aforementioned biological effects. For example, light with a peak wavelength in a range from 400 nanometers (nm) to 450 nm may inactivate microorganisms that are in a cell-free environment and/or inhibit replication of microorganisms that are in a cell-associated environment and/or stimulate enzymatic generation of nitric oxide, while also upregulating a local immune response in target tissue. In this regard, light with a peak wavelength in a range from 400 nm to 450 nm may be well suited for fighting invading viral pathogens and corresponding diseases that may originate in the respiratory tract, including Orthomyxoviridae (e.g., influenza), common colds, coronaviridae (e.g., coronavirus), picornavirus infections, tuberculosis, pneumonia, bronchitis, and sinusitis. In certain embodiments, red or near-infrared (NIR) light (e.g., peak wavelength range from 630 nm to 1,000 nm) may be useful to provide anti-inflammatory effects and/or to promote vasodilation. Anti-inflammatory effects may be useful in treating disorders, particularly microbial disorders that result in inflammation along the respiratory tract. In this regard, red light may be used as part of treatment protocols that reduce any tissue inflammation that may result from exposure to blue light, which may positively impact cell viability, thereby lowering cytotoxicity even further. A decrease in inflammation can be beneficial when treating viral infections, particularly when a virus can elicit a cytokine storm and/or inflammation can result in secondary bacterial infections. Accordingly, the combination of blue light, such as light at around 425 nm, and red light at one or more anti-inflammatory wavelengths, can provide a desirable combination of biological effects.

Depending on the application, other wavelength ranges of light may also be administered to human tissue. For example, UV light (e.g., UV-A light having a peak wavelength in a range of from 315 nm to 400 nm, UV-B light having a peak wavelength in a range of from 280 nm to 315 nm, and UV-C light having a peak wavelength in a range from 200 nm to 280 nm) may be effective for inactivating microorganisms that are in a cell-free environment and/or inhibit replication of microorganisms that are in a cell-associated environment and/or stimulate enzymatic generation of nitric oxide. However, overexposure to UV light may lead to cytotoxicity concerns in associated tissue. It may therefore be desirable to use shorter cycles and/or lower doses of UV light than corresponding treatments with only visible light. In certain embodiments, light with a peak wavelength in a range from 385 nm to 450 nm may be provided to elicit an antimicrobial and/or anti-pathogenic effect. In further embodiments, such wavelengths of light may be used in treatment protocols that also administer anti-inflammatory light. In certain aspects, light sources may be provided with light characteristics in the visible spectrum, for example with light emissions with peak wavelengths primarily in a range from 400 nm to 700 nm. Depending on the target application, light characteristics may also include IR or NIR peak wavelengths at or above 700 nm or UV peak wavelengths at or below 400 nm. As used herein, light may include visual and non-visual electromagnetic radiation with single or multiple peak wavelengths in a range from 180 nm to 4,000 nm.

Doses of light to induce one or more biological effects may be administered with one or more light characteristics, including peak wavelengths, radiant flux, and irradiance to target tissues. Irradiances to target tissues may be provided in a range from 0.1 milliwatts per square centimeter (mW/cm²) to 200 mW/cm², or in a range from 5 mW/cm² to 200 mW/cm², or in a range from 5 mW/cm² to 100 mW/cm², or in a range from 5 mW/cm² to 60 mW/cm², or in a range from 60 mW/cm² to 100 mW/cm², or in a range from 100 mW/cm² to 200 mW/cm². In certain embodiments, irradiances in a range from 0.1 watts per square centimeter (W/cm²) to 10 W/cm² may be safely pulsed to target tissue. Such irradiance ranges may be administered in one or more of continuous wave and pulsed configurations, including light-emitting diode (LED)-based photonic devices that are configured with suitable power (radiant flux) to irradiate a target tissue with any of the above-described ranges.

Light sources may include one or more of LEDs, organic LEDs (OLEDs), lasers, and other lamps according to aspects of the present disclosure. Lasers may be used for irradiation in combination with optical fibers or other delivery mechanisms. LEDs are solid state electronic devices capable of emitting light when electrically activated. LEDs may be configured across many different targeted emission spectrum bands with high efficiency and relatively low costs. Accordingly, LEDs may be used as light sources in photonic devices for phototherapy applications. Light from an LED is administered using a device capable of delivering the requisite power to a targeted treatment area or tissue. High power LED-based devices can be employed to fulfill various spectral and power needs for a variety of different medical applications. LED-based photonic devices described herein may be configured with suitable power to provide irradiances as high as 100 mW/cm² or 200 mW/cm² in the desired wavelength range. An LED array in this device can be incorporated into an irradiation head, hand piece, and/or as an external unit.

In addition to various sources of light, the principles of the present disclosure are also applicable to one or more other types of directed energy sources. As used herein, a directed energy source may include any of the various light sources previously described, and/or an energy source capable of providing one or more of heat, IR heating, resistance heating, radio waves, microwaves, soundwaves, ultrasound waves, electromagnetic interference, and electromagnetic radiation that may be directed to a target body tissue. Combinations of visual and non-visual electromagnetic radiation may include peak wavelengths in a range from 180 nm to 4,000 nm. Illumination devices as disclosed herein may include a light source and another directed energy source capable of providing directed energy beyond visible and UV light. In other embodiments, the other directed energy source capable of providing directed energy beyond visible and UV light may be provided separately from illumination devices of the present disclosure.

Exemplary illumination devices as described herein may be configured to treat tissues within a variety of body cavities. For example, the devices described herein may be configured to treat, prevent, and/or reduce the biological activity of pathogens along tissues of the upper respiratory tract that are accessible via the oral cavity. Such tissues include the pharynx and/or the oropharynx, trachea, and/or esophagus. Representative types of illumination devices described herein may be suitable for positioning at or partially within a patient's mouth for delivery of light to the target tissues.

In certain aspects, illumination devices are disclosed for providing phototherapy in the form of one or more treatment protocols for one or more intended biological effects. As used herein, treatment protocols may also be referred to as light treatment protocols or phototherapy protocols and may include one or more wavelengths of light with corresponding dosing parameters. Depending on the intended biological effect, certain treatment protocols may have limitations on the total amount of dosing required. Certain treatment protocols may vary in dosing and or the administered wavelength of light for a particular biological effect. In certain aspects, illumination devices are disclosed with modular structures that provide the ability to have various changeable attributes, including controlling and/or extending dosing parameters and/or the ability to change administered wavelengths. In this manner, illumination devices are disclosed that may be repeatably modified to tailor light treatments for a variety of biological effects. For example, a modular structure of an illumination device may be configured for treatment of influenza while a different modular structure for the same illumination device may be configured for treatment of coronaviridae.

Modular structures may include tokens, switches, handles, rechargeable power structures, control modules, illumination modules, optics, light guides, and/or light guide positioners, among others. Additionally, the use of modular structures may improve manufacturability of illumination devices and/or reduce overall weights and/or form factors of illumination devices. In certain embodiments, illumination devices may embody connected devices that are part of larger systems that administer and/or monitor light treatment protocols across multiple illumination devices in one or more geographic locations, such as those described in U.S. Publication No. 2022/0212031, the contents of which are incorporated by reference herein in their entirety.

In certain embodiments, modular structures include tokens for providing a controlled number of uses based on a particular treatment protocol. For example, a treatment protocol may involve a certain number of doses to be provided over a certain number of days, after which the illumination device may be programmed to be no longer operable. For additional treatments, a user may replace the existing token of the illumination device with a new one that initiates continued or additional treatments. In certain embodiments, tokens may be provided as a prescription from a medical provider, while in other embodiments, tokens may be provided in over-the-counter form. Switches for initiating operation of the illumination device may be integrated with such tokens in certain embodiments. Modular structures may also include various modules for changing treatment protocols of a particular illumination device, such as illumination modules that provide different wavelengths of light. Additional modular structures may include power structures, such as rechargeable handles and/or communication modules that provide the ability to wireless communication with larger connected systems. Tokens as described above may be integrated within any of the modular structures for initiating and/or controlling treatment protocols.

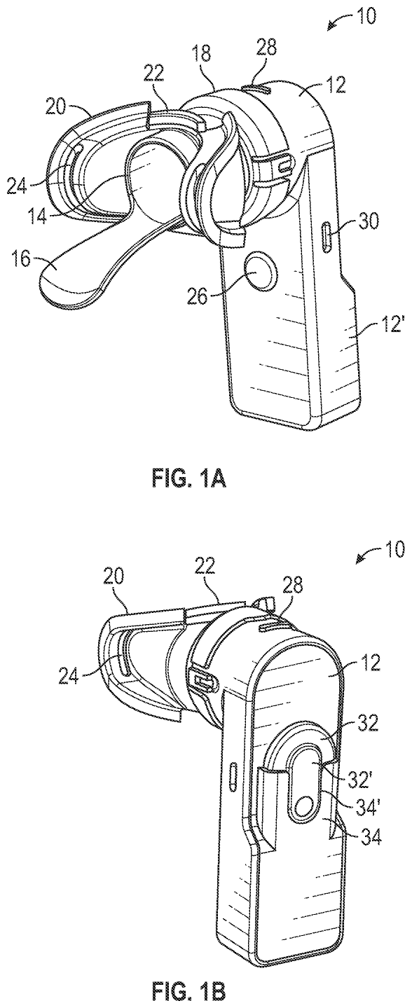

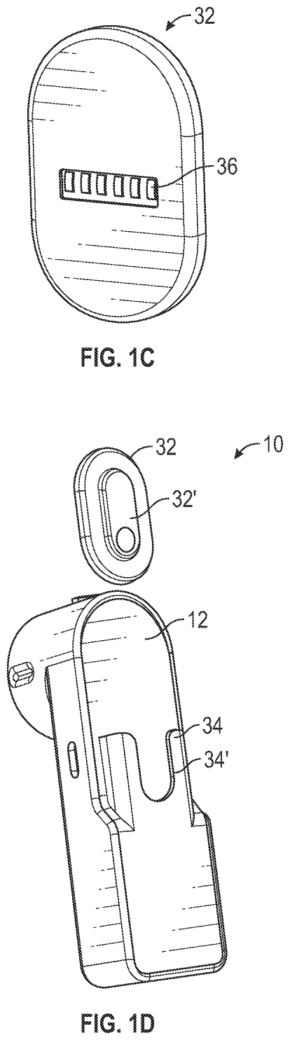

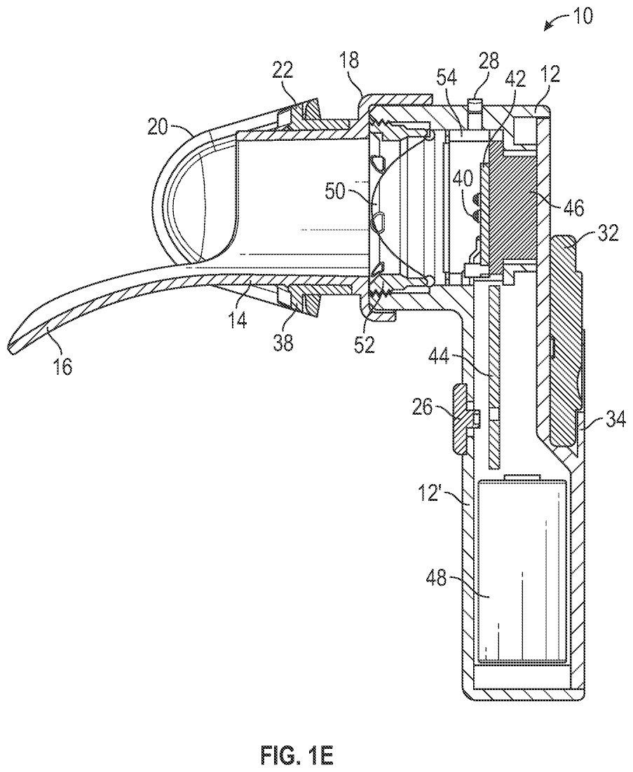

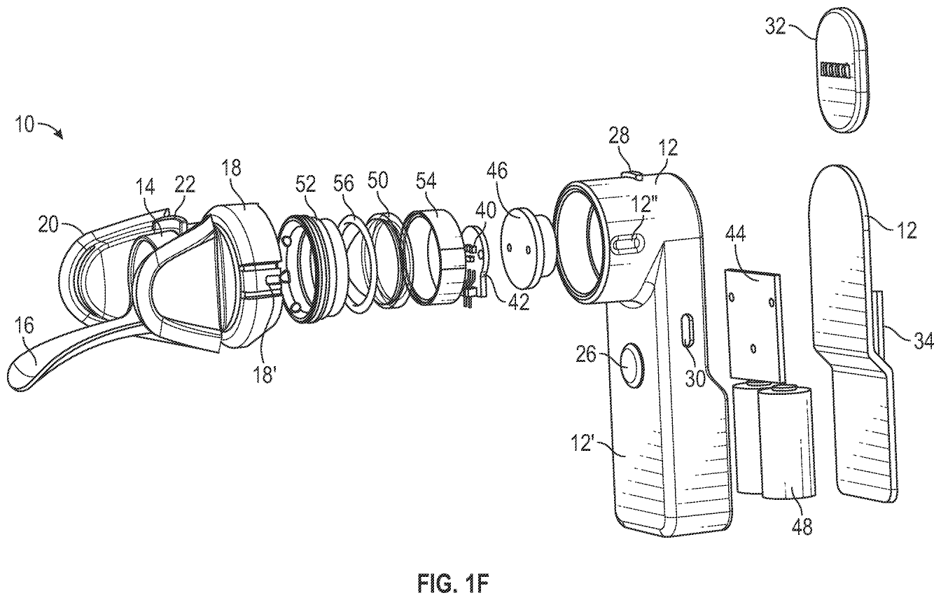

FIG. 1A is front perspective view of an illumination device 10 according to the present disclosure. The illumination device 10 is configured to be a handheld device for positioning at least partially within the oral cavity of a patient (also referred to herein as a “user”). The illumination device 10 includes a housing 12 with one or more light sources, such as LEDs, arranged within the housing 12. In certain embodiments, the housing 12 may comprise a plastic material for light weight applications. In other embodiments, the housing 12 may comprise a metal or a combination of plastic and metal. A light guide 14 is affixed to the housing 12 to direct light source emissions toward a target surface.

The light guide 14 may further include a tongue depressor 16 or analogous feature for displacing a user's tongue when inserted into the user's mouth. The tongue depressor 16 may be formed as an extension of the light guide 14 that is curved downward from the light guide 14. In certain aspects, the tongue depressor 16 is an integral single piece with the light guide 14. The light guide 14 and tongue depressor 16 may be formed of a rigid material, such as rigid plastic materials, to not deform when positioned within the oral cavity. Rather, the light guide 14 and the tongue depressor 16 may serve to displace soft tissue, including the tongue, to provide a more direct path for light to target tissue, such as a back of a user's throat or oropharynx.

In certain embodiments, the light guide 14 forms a hollow core that defines a light-transmissive pathway for propagating light toward a target surface. The hollow core may be formed by light-reflective and/or light-blocking walls of the light guide 14. An exemplary configuration includes hollow cylindrical walls of the light guide 14. In certain aspects, the walls of the light guide 14 may be formed with materials that are white in color to promote light-reflective and/or light-redirecting properties. In further aspects, interior surfaces of the walls of the light guide 14 may be formed with textured or roughened features for redirecting and/or scattering light in various directions as the light propagates along the light guide 14. By arranging the light guide 14 with the hollow core, increased amounts of light may exit the light guide 14 along a direct path without interacting with the walls, while wider-angled light may be reflected and/or redirected by the light guide 14. The light guide 14 may include a flange 18 that facilitates attachment with the housing 12, such as by way of a snap-fit connection.

A light guide positioner 20 may be arranged about a periphery of a portion of the light guide 14. The light guide positioner 20 may be adapted for engaging with one or more surfaces within the oral cavity to secure the illumination device 10 and direct emissions through the light guide 14 in a desired direction. In such embodiments, the light guide positioner 20 may also be referred to as a mouthpiece. In FIG. 1A, a first incisor tab 22 of the light guide positioner 20 is visible at a top of the light guide positioner 20. The first incisor tab 22 may engage with back surfaces of one or more upper incisors of a patient to ensure proper placement and/or alignment of the light guide 14 during use. A second incisor tab may also be provided on a bottom of the light guide positioner 20 for engaging with back surfaces of one or more lower incisors for similar reasons as illustrated in FIG. 1E and described in greater detail below. In certain embodiments, the light guide positioner 20 may comprise a semi-rigid material, such as silicone, that is less rigid than the light guide 14 for improved comfort within the oral cavity. One or more holes 24 may be formed through portions of the light guide positioner 20 to allow air flow for easier breathing during use.

A user interface element 26, such as a tactile element, button, or switch, may be positioned along the housing 12 so that a user may initiate operation of the illumination device 10. The housing 12 may form a handle 12′ for a user to hold during use and the user interface element 26 may be positioned on the handle 12′. In certain embodiments, a window 28 through the housing 12 may be arranged to provide visible indication that the light sources within the housing 12 are illuminated. As used herein, the window 28 may also be referred to as a housing window. This allows the user to know the illumination device 10 is functioning while the light guide positioner 20 and portions of the light guide 14 are inserted into the oral cavity during use. The window 28 may comprise an opening of the housing 12 proximate a location of the internal light source and spaced away from a primary emission surface of the illumination device 10. In the context of the illumination device 10, the primary emission surface is an end of the light guide 14 that is distal to the housing 12. In this regard, the window 28 is spaced away from the light guide positioner 20 and the light guide 14. In certain embodiments, the window 28 may comprise a light transparent material, such as silicone, plastic, or glass. The housing 12 may also include a port 30 that may be a charging port and/or a data transfer port, such as a universal serial bus (USB) port.

FIG. 1B is a back perspective view of the illumination device 10 of FIG. 1A. A token 32 is configured for attachment and detachment with the housing 12. In this manner, the token 32 may also be referred to as a removable token. In certain embodiments, the token 32 is configured for placement along the backside of the housing 12 as illustrated in FIG. 1B, although other locations are also contemplated as later described below. The housing 12 may form a receptacle 34 arranged to receive and secure the token 32 during use. By way of example, the token 32 may include a protrusion 32′ with a shape that corresponds with a shape of a slot 34′ of the receptacle 34. In this manner, the token 32 may slide into place with the receptacle 34. In other examples, other forms of mechanical attachment and/or detachment may be provided. As described above, the token 32 may be configured to initiate a treatment protocol and/or continue a treatment protocol for the illumination device 10. When a particular treatment protocol is completed and/or expired, a user may obtain another token 32 for attachment with the illumination device. Depending on the application and treatment, the token 32 may be obtained by way of a prescription from a health care provider or by way of over-the-counter purchase. In certain embodiments, the token 32 may comprise a physical connection with the illumination device for facilitating transfer of information. In other embodiments, the token 32 may be configured to provide near field communication, such as radio frequency identification (RFID) and/or inductive transfer, with the illumination device 10, in addition to or in lieu of the physical connection.

FIG. 1C is a perspective view of the token 32 of FIG. 1B from a side that faces the housing 12 when positioned within the receptacle 34 of the illumination device 10 of FIG. 1B. As described above, certain examples of the token 32 provide a physical connection for facilitating transfer of information with driver circuitry within the illumination device 10. For example, the token 32 may comprise contacts 36 that connect with corresponding connections within the receptacle 34 of FIG. 1B. The contacts 36 may embody a variety of electrical connections, including pins for pinout connections. As described above, the token 32 may be configured for near field communication. In such embodiments, the contacts 36 may still be present to also provide the physical connections in combination with near field communication or the contacts 36 may be omitted. FIG. 1D is a back perspective view of the illumination device 10 of FIGS. 1A and 1B illustrating removable attachment capabilities of the token 32. For illustrative purposes, the light guide 14 and the light guide positioner 20 of FIGS. 1A and 1B are omitted. As illustrated in FIG. 1D, the token 32 may be configured for removable attachment with the housing 12 by way of the receptacle 34. In this manner, once a particular treatment protocol is completed, the token 32 may be disposed of and replaced with another token 32 that controls operation of the illumination device 10.

FIG. 1E is a cross-sectional view of the illumination device 10 of FIGS. 1A and 1B. As illustrated, the light guide positioner 20 may comprise a second incisor tab 38 on an opposing side of the light guide 14 from the first incisor tab 22, such as the top side and the opposing bottom side of the light guide 14. When inserted into the oral cavity, the first incisor tab 22 may engage with back surfaces of one or more upper incisors of a patient, and the second incisor tab 38 may engage with back surfaces of one or more lower incisors of the patient. Accordingly, the light guide positioner 20 and incisor tabs 22, 38 may secure portions of the light guide 14 at an intended position for phototherapy. In certain embodiments, the first incisor tab 22 may be arranged closer to the housing 12 and/or the flange 18 of the light guide 14 than the second incisor tab 38. In this manner, when the first and second incisor tabs 22, 38 are respectively engaged with upper and lower incisors of a patient, the emission direction through the light guide 14 will be tilted downward relative to the oral cavity in a direction that targets the various portions of the pharynx, including the oropharynx. Additionally, the tongue depressor 16 may also be tilted downward to apply increased pressure on the user's tongue to further expose the oropharynx for receiving light. Notably, such a configuration may provide increased irradiance to the oropharynx when the lower jaw of a patient is opened to receive the light guide positioner 20. Further details regarding positioning of the incisor tabs 22, 38 may be found in U.S. patent application Ser. No. 18/184,129, the contents of which are incorporated by reference herein in their entirety.

As illustrated in the cross-sectional view of FIG. 1E, one or more light sources 40 are arranged within the housing 12. In certain embodiments, the light sources 40 may be arranged on a first board 42, such as a printed circuit board, and electrically coupled with driver circuitry 44 of a second board or printed circuit board. In certain embodiments, the light sources 40 and first board 42 may be positioned against and in thermal communication with a heatsink 46. As used herein, the heatsink 46 may also be referred to as a heatsink puck. As illustrated, the heatsink 46 resides within the housing 12 to internally draw heat away from the light sources 40 during operation. An energy storage device 48, such as a rechargeable battery, may be positioned within the housing 12.

The illumination device 10 may further include a lens 50 positioned between the light guide 14 and the one or more light sources 40. The lens 50 may be useful for controlling and directing an emission pattern from the one or more light sources 40 into the light guide 14. In certain embodiments, a portion of the lens 50, such as an apex of the lens 50, may at least partially reside within the light guide 14 to further direct emissions. A lens retainer 52 may further be arranged between the light guide 14 and the one or more light sources 40 to hold the lens 50 in place. In certain embodiments, a spacer 54 may be arranged to extend between the lens 50 and the one or more light sources 40 to position the lens 50 at a suitable distance. In this manner, the lens 50 may be sandwiched between the lens retainer 52 and the spacer 54. In certain embodiments, the spacer 54 may have an opening that is aligned with the window 28 for providing visual indication of when the one or more light sources 40 are electrically activated. In this manner, the window 28 is positioned to received light emitted from the one or more light sources 40 that is offset from a primary emission direction through the light guide 14, such as at least 45 degrees or in a range of 45 degrees to 90 degrees offset from the primary emission direction.

FIG. 1F is an exploded view of the illumination device 10 of FIG. 1E. As illustrated, a gasket 56, such as an O-ring, may be assembled between the lens 50 and the lens retainer 52. In certain embodiments, the housing 12 may comprise two separate parts that are joined together. For example, in the exploded view, a portion of the housing 12 is illustrated to be connected to the remainder of the housing 12 to enclose the energy storage device 48. In certain embodiments, the receptacle 34 may be incorporated with this portion of the housing 12 for receiving the token 32. As further illustrated in FIG. 1F, the housing 12 may form one or more indentations 12″ that are configured to receive the light guide 14. For example, tabs 18′ of the flange 18 may snap-fit with corresponding indentations 12″ to secure the light guide 14 and light guide positioner 20 in place.

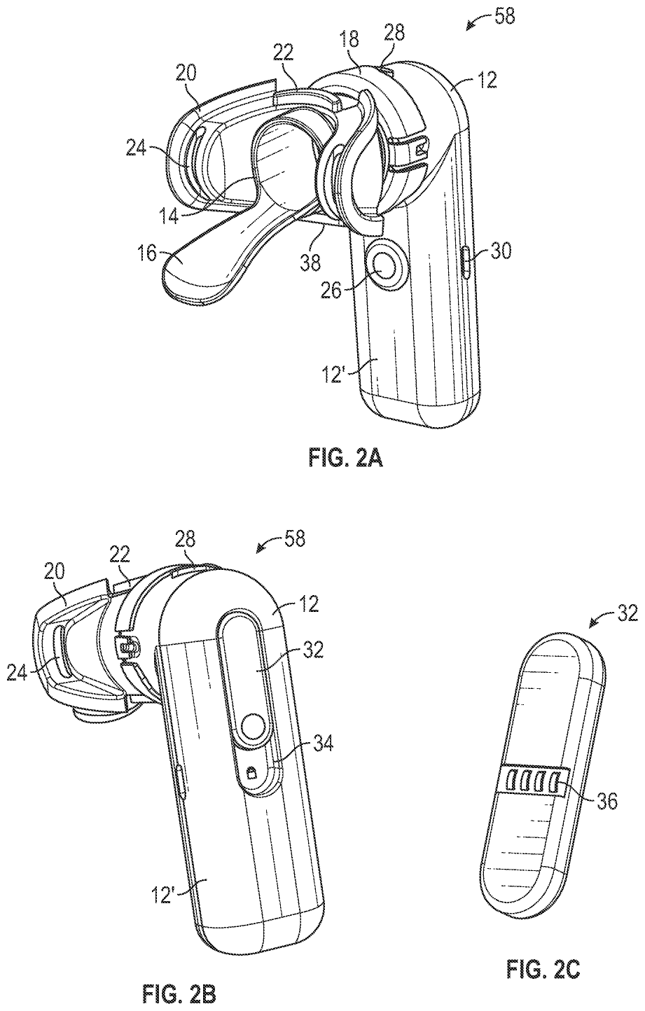

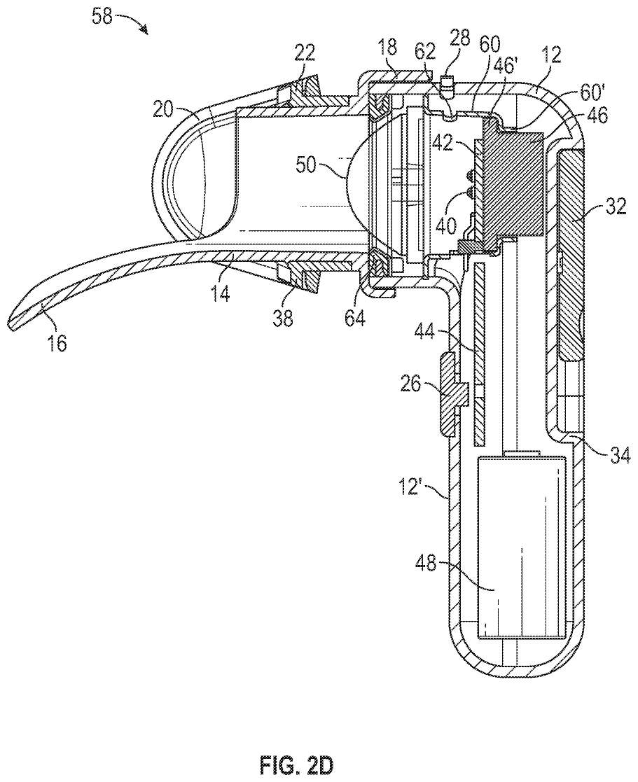

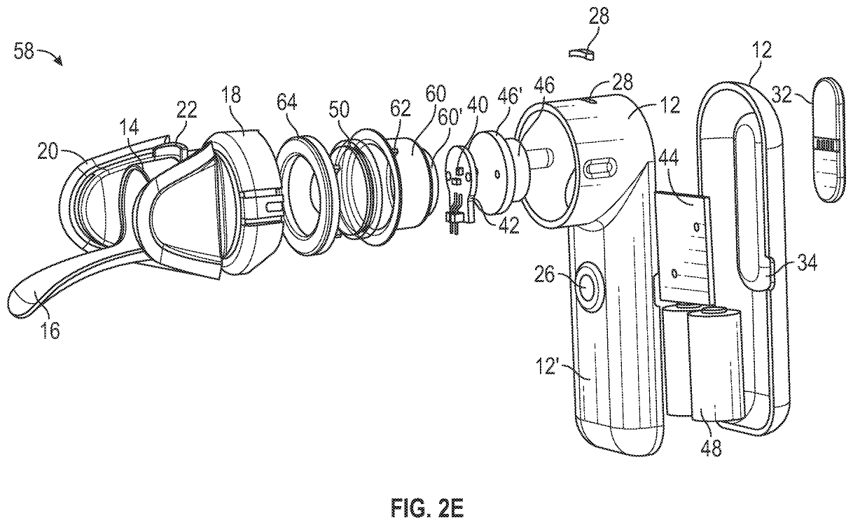

FIG. 2A is a front perspective view of an illumination device 58 that is similar to the illumination device 10 of FIG. 1A. FIG. 2B is a back perspective view of the illumination device 58 of FIG. 2A illustrating a different arrangement of the token 32 and receptacle 34 as compared with the illumination device 10 illustrated in FIG. 1B. FIG. 2C is a perspective view of the token 32 of FIG. 2B. In FIG. 2B, the receptacle 34 forms a recess within a portion of the housing 12. The receptacle 34 is formed with a shape that corresponds with a shape of the token 32. By way of example, the token 32 in FIG. 2B forms an elongated shape with rounded ends. In this regard, the receptacle 34 also forms an elongated shape with rounded ends configured to receive the token 32. The token 32 may be press-fit or snap-fit into the receptacle 34. As illustrated, the receptacle 34 may have a shape that allows the token to slide within the receptacle 34 to lock in place.

FIG. 2D is a cross-sectional view of the illumination device 58 of FIGS. 2A and 2B. In certain embodiments, the internal structure of the illumination device 58 may be the same as the illumination device 10 as illustrated in FIG. 1E. In other embodiments, the illumination device 58 may include an optical cup 60 that is arranged between the lens 50 and the heatsink 46. As illustrated, a portion 60′ of the optical cup 60 may be configured to attach with a protruding lip 46′ of the heatsink 46. For example, the portion 60′ of the optical cup 60 that attaches to the heatsink 46 may comprise an inward bend or clip that conforms to the shape of the protruding lip 46′. In this manner, the heatsink 46 may be retained by the optical cup 60 and a remaining portion of the heatsink 46 may extend away from the optical cup 60 and the one or more light sources 40. As illustrated, the protruding lip 46′ may extend from the heatsink 46 in a direction parallel with a mounting surface for the light sources 40 and/or first board 42. As further illustrated, the optical cup 60 may have an opening 62 that is aligned or otherwise registered with the window 28 to permit off angle light to reach the window 28. The optical cup 60 may peripherally surround the light sources 40 to direct light in a targeted emission direction toward the lens 50 and light guide 14. In certain embodiments, the optical cup 60 may have interior walls that angle outward in a direction from the heatsink 46 toward the lens 50 to further direct light from the light sources 40. In FIG. 2D, a gasket 64 is illustrated between the lens 50 and the optical cup 60. The gasket 64 may embody a plastic lens retainer with an over-molded gasket.

FIG. 2E is an exploded view of the illumination device 58 of FIG. 2D. As illustrated, the housing 12 may comprise separate parts that are joined together in the final assembly. In a similar manner as described above for the illumination device 10 of FIG. 1F, a portion of the housing 12 may attach to the remainder of the housing 12 to enclose the energy storage device 48 and form the receptacle 34 for the token 32. In the exploded view, the window 28 is illustrated as a combination of an opening in the housing 12 and a transparent material, such as silicone, that fits within the opening of the housing 12. In certain embodiments, the optical cup 60 and the gasket 64 as described for the illumination device 58 may replace the lens retainer 52, the spacer 54, and the gasket 56 in the illumination device 10 of FIGS. 1A to 1F.

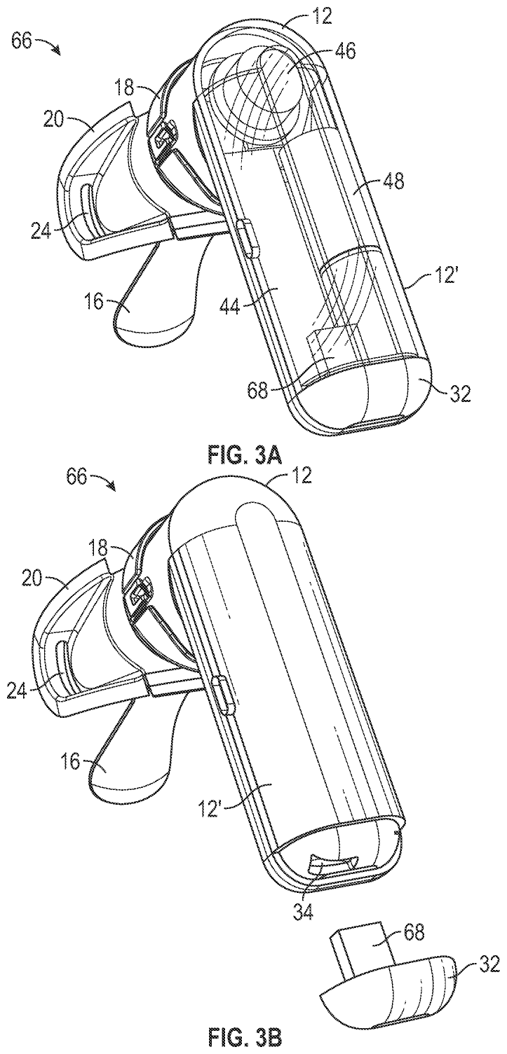

FIG. 3A is a back perspective view of an illumination device 66 that is similar to the illumination device 58 of FIG. 2B except the token 32 is attached to a base of the housing 12. For illustrative purposes, the housing 12 is shown as transparent in FIG. 3A to show locations of the heatsink 46, the energy storage device 48, and the driver circuitry 44. In certain embodiments, the token 32 comprises a plug 68 that is inserted into the housing 12 when the token 32 is attached. The plug 68 may be electrically coupled with the driver circuitry 44 when the token 32 is attached to the handle 12′. As illustrated, the token 32 may form a base of the handle 12′ of the illumination device 66. FIG. 3B is a back perspective view of the illumination device 66 of FIG. 3A with the token 32 illustrated as detached from the housing 12. In FIG. 3B, the housing 12 is illustrated without transparency. As illustrated, the receptacle 34 of the housing 12 is shaped to receive the plug 68 of the token 32. In certain embodiments, the plug 68 may form a USB connection with the receptacle 34.

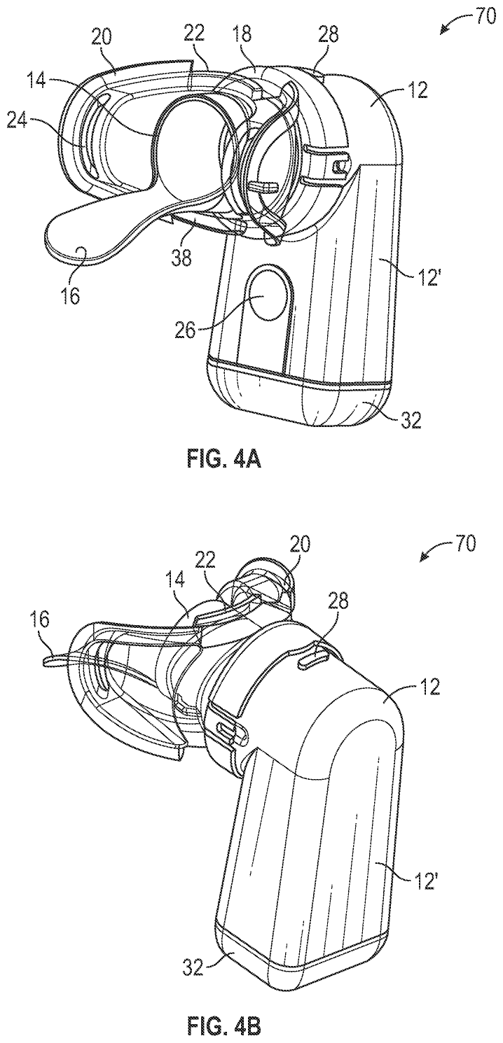

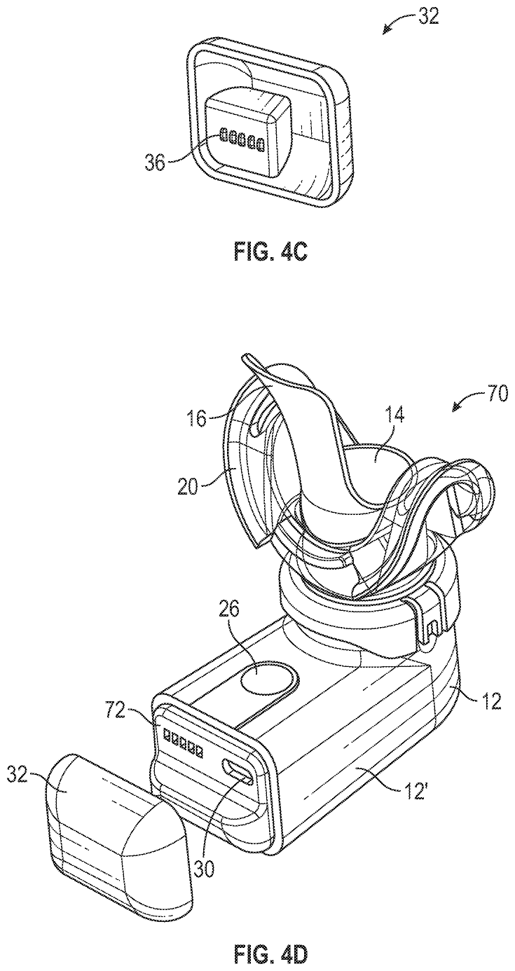

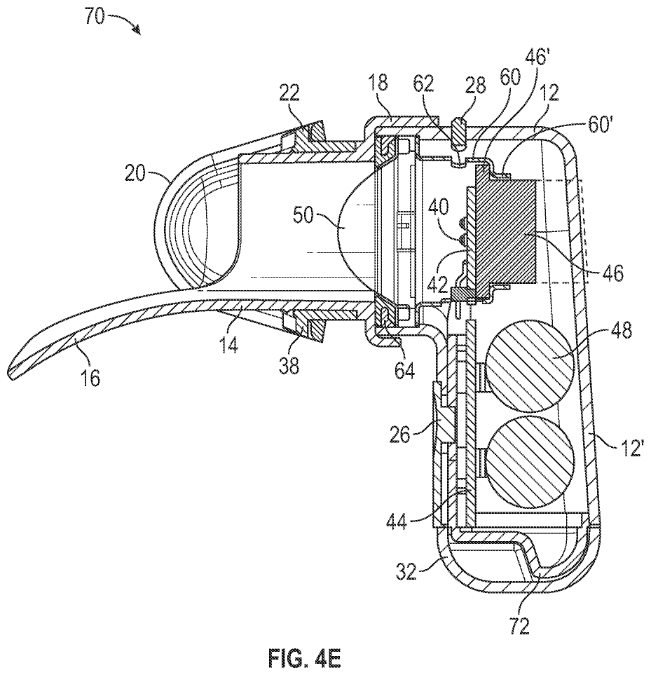

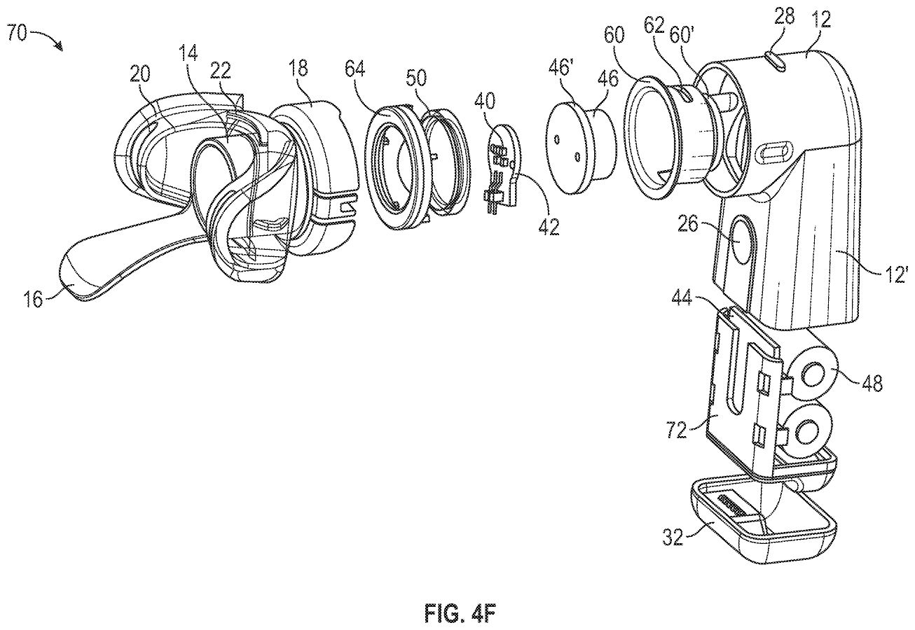

FIG. 4A is a front perspective view of an illumination device 70 that is similar to the illumination device 66 of FIGS. 3A and 3B with a different attachment structure for the token 32. FIG. 4B is a back perspective view of the illumination device 70 of FIG. 4A. FIG. 4C is a perspective view of the token 32 of FIG. 4A. FIG. 4D is a perspective view of the illumination device 70 of FIG. 4A with the token 32 illustrated in exploded view relative to the housing 12. As illustrated, the illumination device 70 may comprise a carriage 72 that fits within the housing 12 in a location where the token 32 is attached. In certain embodiments, the token 32 may be configured to make a snap-fit connection with the carriage 72 and/or housing 12. In FIG. 4D, the token 32 is illustrated for attachment with a base of the housing 12 that forms the handle 12′ of the illumination device 70. In certain embodiments, the charging port 30 may be positioned at the carriage 72 such that the token 32 encloses the charging port 30 during use.

FIG. 4E is a cross-sectional view of the illumination device 70 of FIGS. 4A to 4D, and FIG. 4F is an exploded view of the illumination device 70 of FIGS. 4A to 4D. As illustrated, the carriage 72 extends into the housing 12 from a base of the handle 12′. In certain embodiments, the driver circuitry 44 and the energy storage device 48 may be attached to the carriage 72. For example, the carriage 72 may comprise a slot for receiving the driver circuitry 44 and one or more recesses or slots for receiving the energy storage device 48. The carriage 72 may be configured to be snap-fit within the housing 12. As best illustrated in FIG. 4E, such a configuration may provide the illumination device 70 with a more compact form factor than previous embodiments. In certain embodiments, the compact form factor may also provide additional space within the housing 12 proximate the heatsink 46. This allows the heatsink 46 to have increased size, particularly in a direction extending away from the one or more light sources 40. In certain embodiments, the heatsink 46 could even extend through a back surface of the housing 12 to be exposed to an exterior as illustrated by the superimposed dashed lines in FIG. 4E. In such embodiments, the heatsink 46 may even be threaded to screw through the portion of the housing 12 at the superimposed dashed lines.

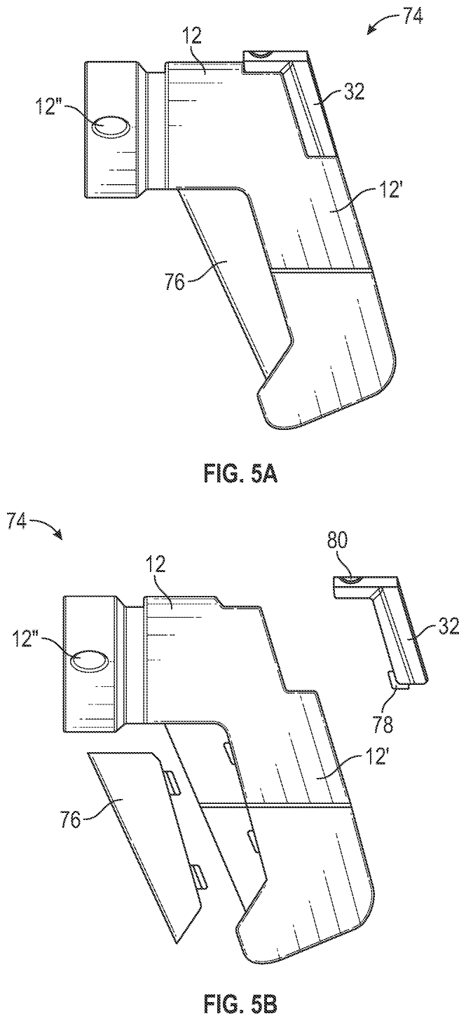

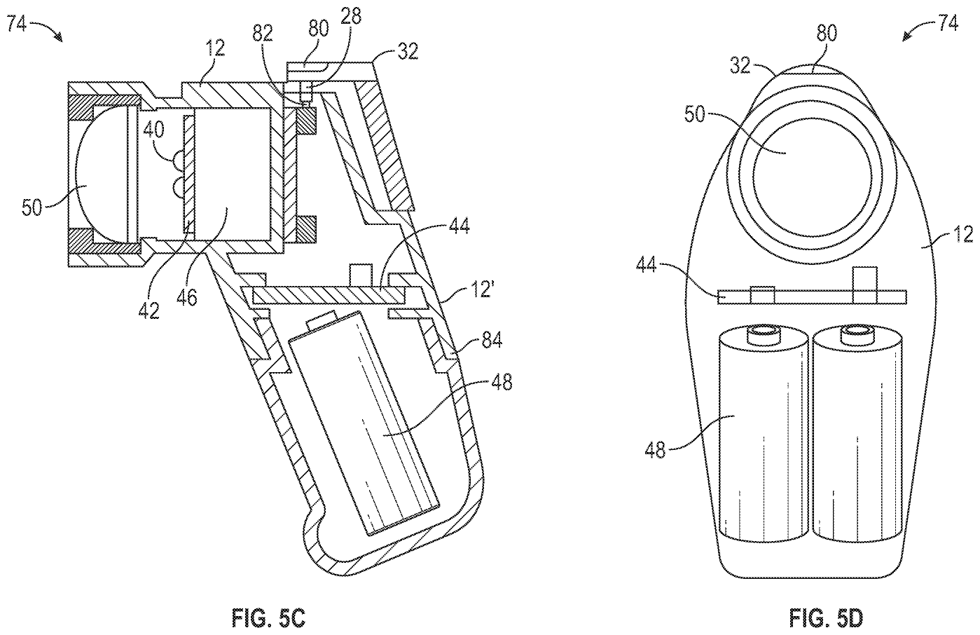

FIG. 5A is a side view of an illumination device 74 that is similar to the illumination device 10 of FIG. 1A except the token 32 also forms a switch for initiating operation of the illumination device 74. In this regard, the user interface element 26 of FIG. 1A may be omitted. In certain embodiments, a grip 76 may be attached to the housing 12. The grip 76 may have an identifier, such as a color or text, that corresponds with a particular wavelength or treatment protocol that the illumination device 74 is configured to provide. For example, the grip 76 may have a first color if the illumination device 74 is configured to treat influenza and the grip 76 may have a different second color if the illumination device 74 is configured to treat coronaviridae. In certain embodiments, the housing 12 may form one or more of the indentations 12″ for attachment of the light guide 14 and light guide positioner 20 of FIG. 1A. As illustrated, the handle 12′ of the housing 12 may be angled away from the primary emission direction. In the context of light treatments for the oral cavity, the handle 12′ may accordingly be positioned farther away from a user's chin.

FIG. 5B is an exploded view of the illumination device 74 of FIG. 5A. When the token 32 is attached to the housing 12, the token 32 may also form a button for activating the illumination device 10. The token 32 may comprise an electrical connector 78 that is electrically coupled with driver circuitry internal to the housing 12. In certain embodiments, a token window 80 may be provided with the token to provide visual indication when the illumination device is electrically activated. Further details are described below for FIG. 5C.

FIG. 5C is a cross-sectional view of the illumination device 74 of FIG. 5A. In certain embodiments, an additional light source 82 may be positioned away from the one or more light sources 40 that provide the primary emission for the illumination device 74. The additional light source 82 may be positioned within the housing 12 and registered with the window 28. As illustrated, the window 28 may in turn be registered with the token window 80 to provide visual indication when the illumination device 74 is turned on. In certain embodiments, the additional light source 82 may comprise an LED. In certain embodiments, the housing 12 may form multiple parts that are fastened together at a seam 84, thereby allowing access and/or replacement of the energy storage device 48. As further illustrated, the driver circuitry 44 may be secured within one or more slots or channels of the housing 12.

FIG. 5D is a front view of the illumination device 74 of FIG. 5C with the housing 12 represented as transparent for illustrative purposes. As illustrated, the lens 50 forms a primary emission surface out of a front of the illumination device 74 while the token window 80 provides visual indication through a top of the illumination device 74.

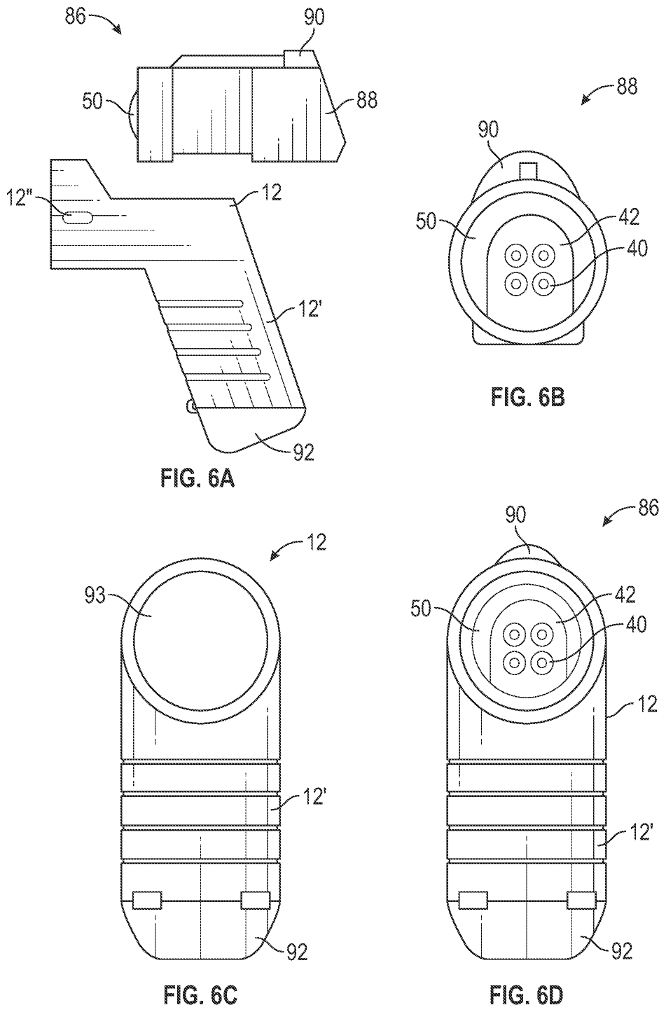

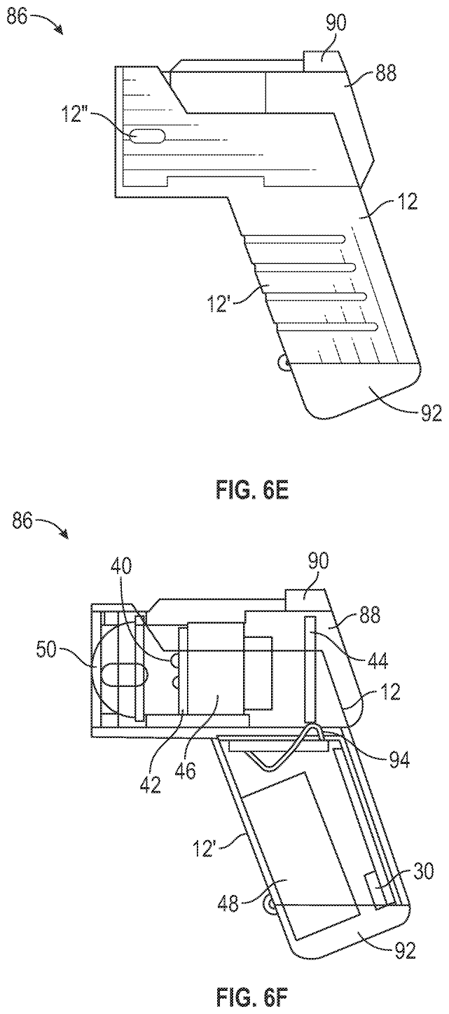

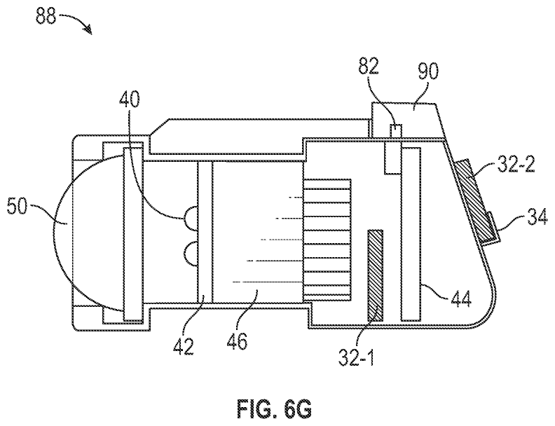

FIG. 6A is an exploded side view of an illumination device 86 that is similar to the illumination device 74 of FIGS. 5A to 5D except the illumination device 86 includes an illumination cartridge 88 for attachment with the housing 12. In this regard, the illumination cartridge 88 may be changeable with regard to the housing 12 to provide different emission wavelengths and/or treatment protocols. As such, the illumination cartridge 88 may include the token and/or take the place of the token 32 as described in previous embodiments. That is, the illumination cartridge 88 may be prescribed by a health care provider or obtained over-the-counter for attachment with the housing 12 to induce a particular biological effect. The illumination cartridge 88 may embody a disposable structure that is replaced for implementation of additional treatment protocols. In certain embodiments, the illumination cartridge 88 includes a cartridge window 90 configured to provide visual indication when the illumination device 86 is turned on. The cartridge window 90 may also serve as a switch or button for a user to initiate operation. The illumination device 86 may further include a door 92 at a base of the handle 12′ that will be described below in more detail.

FIG. 6B is a front view of the illumination cartridge 88 of FIG. 6A. As illustrated, the one or more light sources 40 and the first board 42 are visible through the lens 50, all of which are incorporated within the illumination cartridge 88. The cartridge window 90 is arranged to protrude above a remainder of the illumination cartridge 88 for easier viewing during operation. FIG. 6C is a front view of the housing 12 of the illumination device 86 of FIG. 6A. As illustrated, the housing forms an opening 93 that is sized to receive the illumination cartridge 88 of FIG. 6B, thereby forming a primary emission surface at the opening 93. FIG. 6D is a front view of the illumination device 86 of FIG. 6A with the illumination cartridge 88 in place. The illumination cartridge 88 may slide into place along an upper portion of the housing 12 to lock in place. As illustrated, the lens 50 fits within the opening 93 of FIG. 6C to position the one or more light sources 40 to emit light in the primary emission direction. FIG. 6E is a side view of the illumination device 86 of FIG. 6A with the illumination cartridge 88 attached in place. In certain embodiments, the illumination cartridge 88 may be configured to snap-fit to the housing 12. In certain embodiments, the housing 12 may form one or more of the indentations 12″ for attachment of the light guide 14 and light guide positioner 20 of FIG. 1A.

FIG. 6F is a cross-sectional view of the illumination device 86 of FIG. 6E. As illustrated, the port 30 may be accessible by way of the door 92. In this regard, the port 30 may be enclosed by the door 92 during use. In certain embodiments, the driver circuitry 44 for the one or more light sources 40 is positioned within the illumination cartridge 88. In this manner, each illumination cartridge 88 that may be releasably attached is integrated with the one or more light sources 40 and associated driver circuitry 44. In other embodiments, the driver circuitry 44 may reside within other portions of the housing 12 so that the driver circuitry 44 is not changed when the illumination cartridge 88 is replaced. The housing 12 comprises one or more electrical connectors 94 configured to make electrical connections with the driver circuitry 44 when the illumination cartridge 88 is snapped into place.

FIG. 6G is a cross-sectional view of the illumination cartridge 88 of FIG. 6F. As illustrated, the illumination cartridge 88 includes the lens 50, the one or more light sources 40, and the driver circuitry 44. The illumination cartridge 88 may also comprise the heatsink 46. In certain embodiments, the illumination cartridge 88 may comprise the additional light source 82 positioned to provide indicator light to the cartridge window 90. The additional light source 82 may be electrically coupled to a perimeter of the board of the driver circuitry 44. As described above, the illumination cartridge 88 may include the token 32 and/or take the form of the token 32 as described for previous embodiments. The token 32 may be integrated within the illumination cartridge 88 or the token 32 may be removable from the illumination cartridge. For illustrative purposes, FIG. 6G shows the token labeled 32-1 as integrated within the illumination cartridge 88. In the alternative configuration, FIG. 6G shows the token labeled 32-2 as being removable from the illumination cartridge 88. In such alternative embodiments, the illumination cartridge 88 may include the receptacle 34 as previously described and the token labeled 32-1 may be omitted.

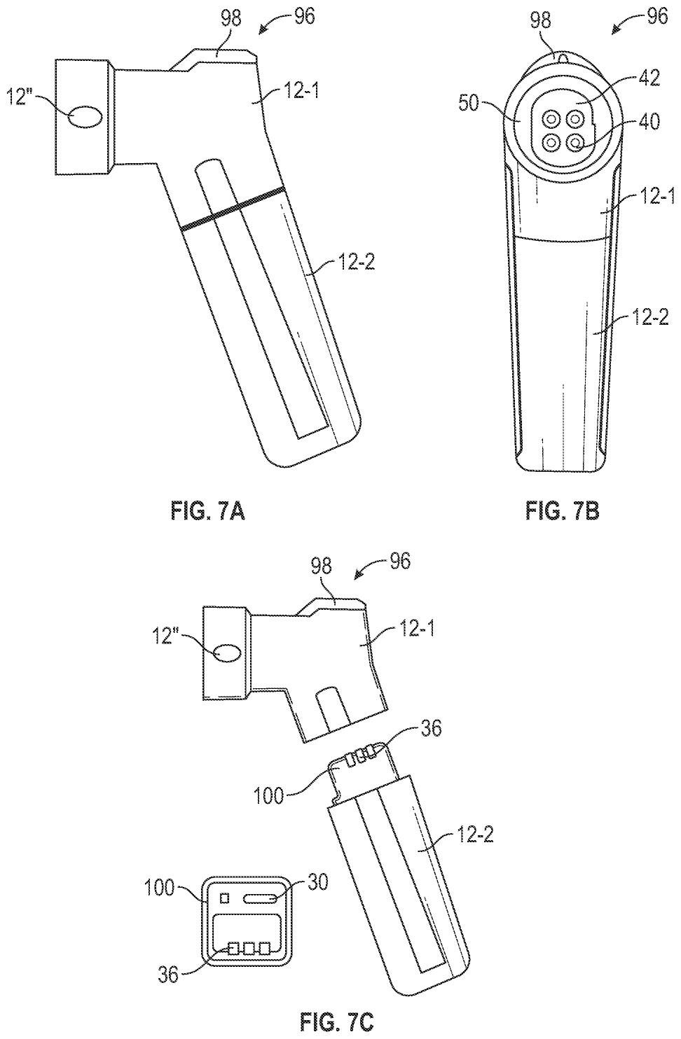

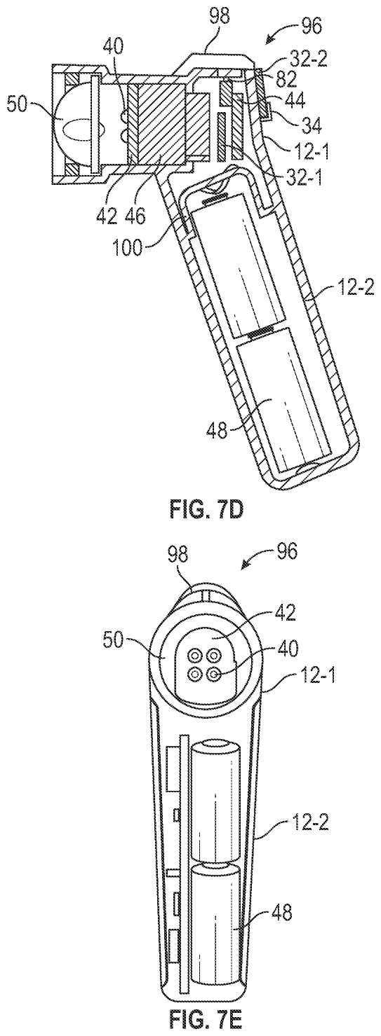

FIG. 7A is an exploded side view of an illumination device 96 that is similar to the illumination device 86 of FIGS. 6A to 6G for embodiments where the housing 12 of previous embodiments comprises an illumination head 12-1 and a detachable handle 12-2. FIG. 7B is a front view of the illumination device 96 of FIG. 7A. The illumination head 12-1 may include many of the elements of the illumination cartridge 88 of FIGS. 6A to 6G, just with a form factor that facilitates attachment with the handle 12-2. In this regard, the one or more light sources 40, the first board 42, and the lens 50 may be present within the illumination head 12-1. An illumination head window 98 may serve a similar purpose to the cartridge window 90 of FIG. 6A. That is, the illumination head window 98 may be configured to provide visual indication when the illumination device 96 is electrically activated and serve as a switch or button for a user to initiate operation. In certain embodiments, the illumination head 12-1 may form one or more of the indentations 12″ for attachment of the light guide 14 and light guide positioner 20 of FIG. 1A. In certain embodiments, the illumination head 12-1 may include or take the place of the token 32 of previous embodiments.

FIG. 7C is a side view of the illumination device 96 of FIG. 7A with the handle 12-2 detached from the illumination head 12-1. A connector 100 may be positioned at a top of the handle 12-2 for providing electrical connection with the illumination head 12-1. The connector 100 may comprise contacts 36 and a port 30 as previously described. By placing the port 30 within the handle 12-1, the handle 12-1 may embody a rechargeable handle for use with one or more different types of illumination heads 12-1. In this manner, different types of the illumination head 12-1 may be prescribed by a health care provider or obtained over-the-counter for attachment with the handle 12-2 for phototherapy to induce a particular biological effect. Alternatively, the handle 12-2 may further embody the token 32 as previously described for FIGS. 1A to 1F. That is, the handle 12-2 may be obtained by way of a prescription from a health care provider or obtained over-the-counter for phototherapeutic treatments.

FIG. 7D is a cross-sectional view of the illumination device 96 of FIG. 7A. As illustrated, the connector 100 may be configured for attachment, such as a snap or press-fit connection, with the illumination head 12-1. The connector 100 may also facilitate electrical connections between the energy storage device 48 and the driver circuitry 44. The illumination head 12-1 may include the additional light source 82 for providing visual indication when the illumination device 96 and the one or more light sources 40 are electrically activated. In this manner, light from the additional light source 82 may be directed through the illumination head window 98. FIG. 7E is a front view of the illumination device 96 of FIG. 7D with portions of the illumination head 12-1 and the handle 12-2 represented as transparent for illustrative purposes. From this perspective, light from the one or more light sources 40 may be directed through the lens 50 in a primary emission direction, such as within an oral cavity. As described above, the illumination head window 98 may also serve as a button to initiate operation once the illumination device 96 is positioned for use. Particularly for oral cavity applications, the illumination head window 98 is suitably positioned for the user to view visual indication when the one or more light sources 40 are electrically activated. As described above, the illumination head 12-1 may include the token 32 and/or take the form of the token 32 as described for previous embodiments. For illustrative purposes, FIG. 7D shows the token labeled 32-1 as integrated within the illumination head 12-1. In an alternative configuration, FIG. 7D shows the token labeled 32-2 as being removable from the illumination head 12-1. In such alternative embodiments, the illumination head 12-1 may include the receptacle 34 as previously described and the token labeled 32-1 may be omitted.

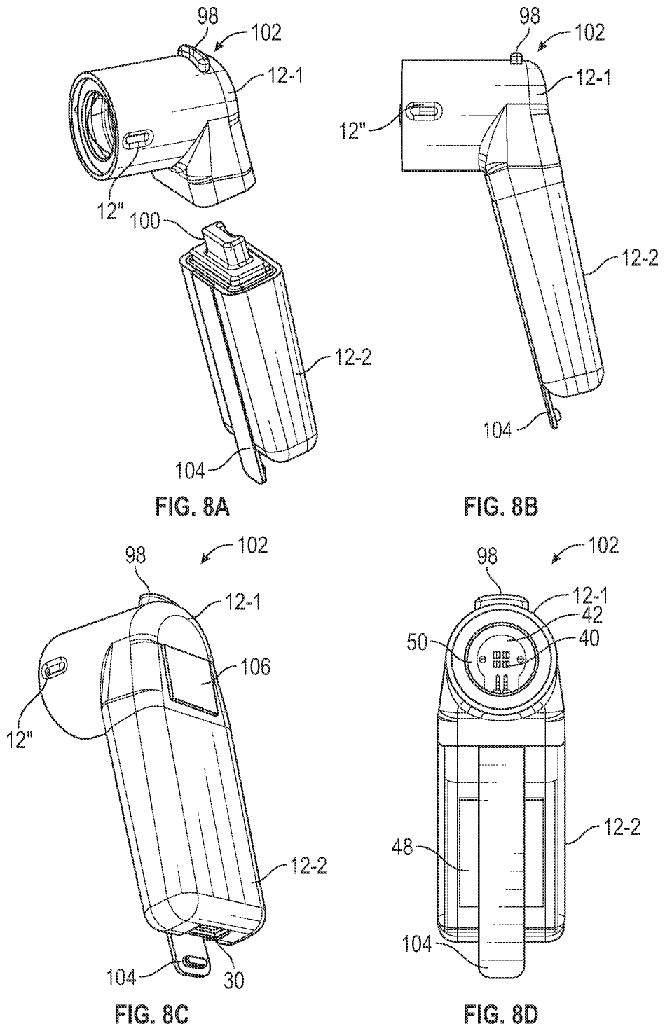

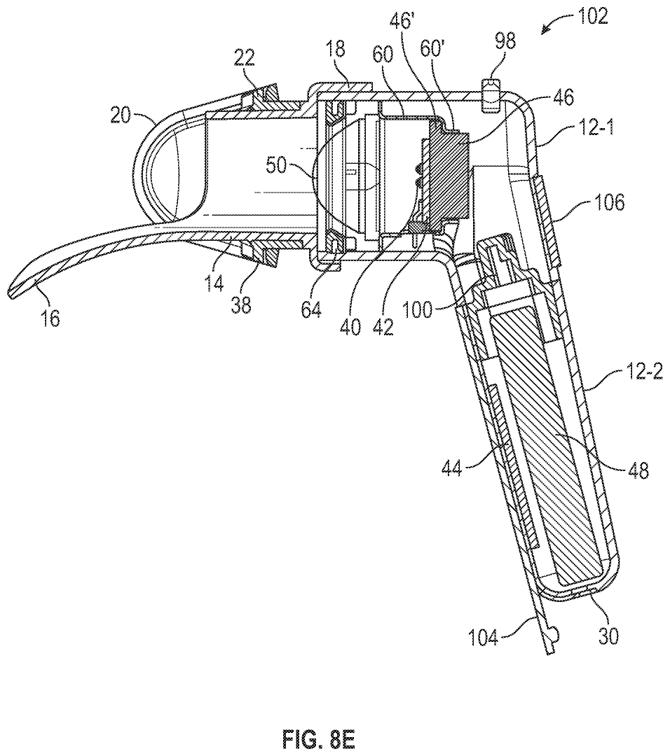

FIG. 8A is a side view of another embodiment of an illumination device 102 that is similar to the illumination device 96 of FIG. 7C with the handle 12-2 detached from the illumination head 12-1. FIG. 8B is a side view of the illumination device 102 of FIG. 8A with the handle 12-2 attached to the illumination head 12-1. FIG. 8C is a back perspective view of the illumination device 102 of FIG. 8A. FIG. 8D is a front view of the illumination device 102 of FIG. 8C with a portion of the handle 12-2 shown as transparent for illustrative purposes.

As best illustrated in FIG. 8C, the port 30 of the illumination device 102 is positioned at a base of the handle 12-2. In this manner, the handle 12-2 does not have to be detached from the illumination head 12-1 for charging. In certain embodiments, the handle 12-2 may include a cover 104 that covers the port 30 when the illumination device 102 is not being charged. For example, the cover 104 may embody a flexible material, such as silicone, that is provided on the handle 12-2 such that a portion of the cover 104 may bend and be pressed into the port 30.

As further illustrated in FIG. 8C, the illumination device 102 may further comprise a user interface display 106 on a backside of the illumination head 12-1. The user interface display 106 may provide various information to the user, such as a battery charge level and/or treatment-related information. In certain embodiments, the treatment-related information may comprise a specific type or treatment and/or intended biological effect, a prescribed dosing, and/or a number of treatments remaining according to an intended treatment protocol. In certain embodiments, the user interface display 106 may also function as a button or switch for initiation operation of the illumination device 102. By positioning the switch along a backside of the illumination device 102, a user may avoid unintentional powering off during use. For example, in oral cavity applications, the front side of the handle 12-2 may be positioned proximate a user's chin. With electrical activation capabilities moved to the backside, the user's chin is not able to accidentally press the button and turn off the device. As described here, the user interface display 106 as described for FIG. 8C may be implemented on any of the previously described embodiments, including those illustrated by FIGS. 1A to 1F, FIGS. 2A to 2E, FIGS. 3A to 3B, FIGS. 4A to 4F, FIGS. 5A to 5D, FIGS. 6A to 6G, and FIGS. 7A to 7E. In still further embodiments, the illumination head window 98 may serve as the button or switch for turning the illumination device 102 on and off as previously described.

FIG. 8E is a cross-sectional view of the illumination device 102 of FIG. 8A with the addition of the light guide 14 and light guide positioner 20. As illustrated, the light guide 14 and light guide positioner 20 may be attached to the illumination head 12-1 for a targeted application, such as within the oral cavity. As further illustrated, the illumination head 12-1 may include the optical cup 60 and heatsink 46 as described above for the illumination device 58 of FIG. 2D. In certain embodiments, the driver circuitry 44 may be positioned within the handle 12-2 as shown. Alternatively, the driver circuitry 44 may be positioned within the illumination head 12-1 as illustrated for the illumination device 96 of FIG. 7D. In certain embodiments, the illumination head 12-1 may include the token (32-1 or 32-2) as illustrated in FIG. 7D.

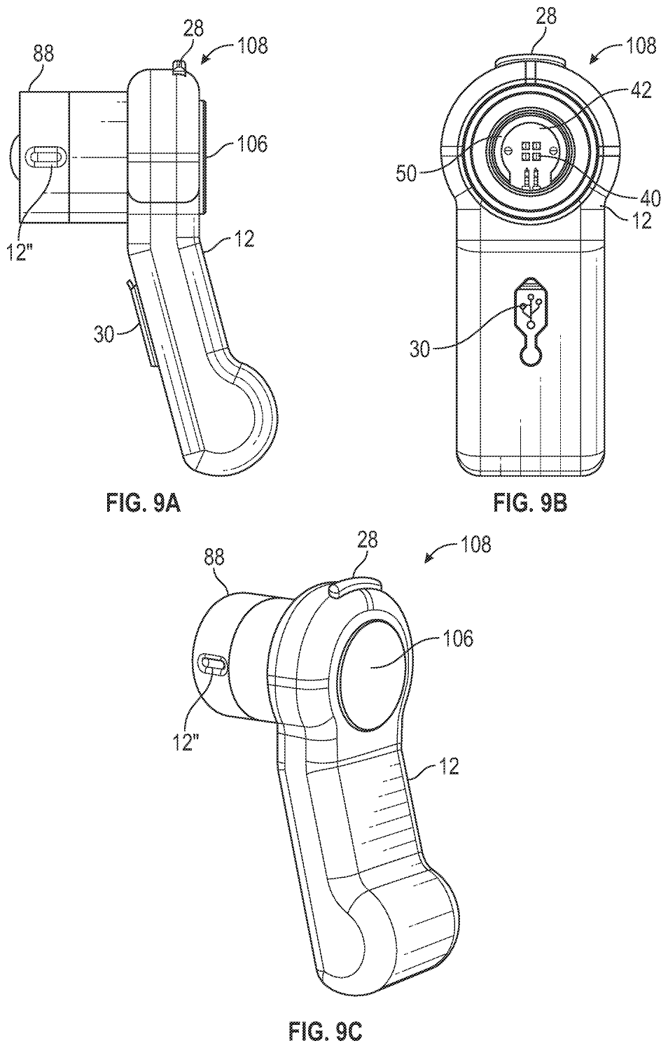

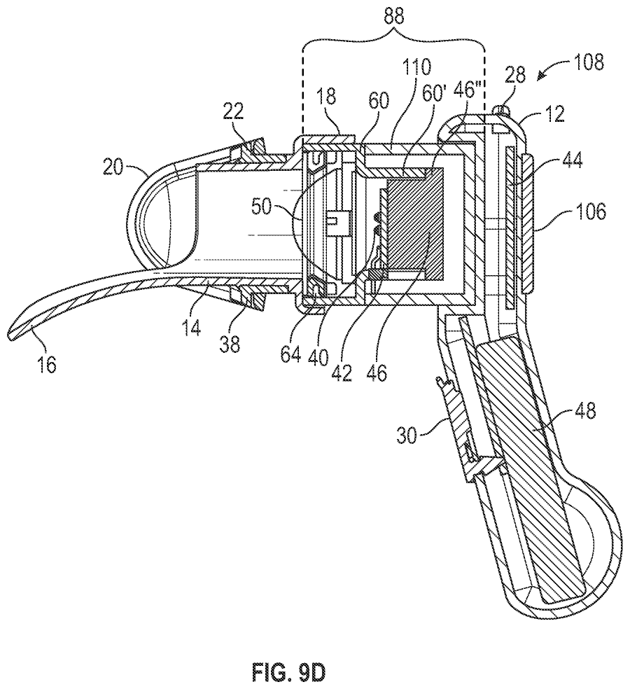

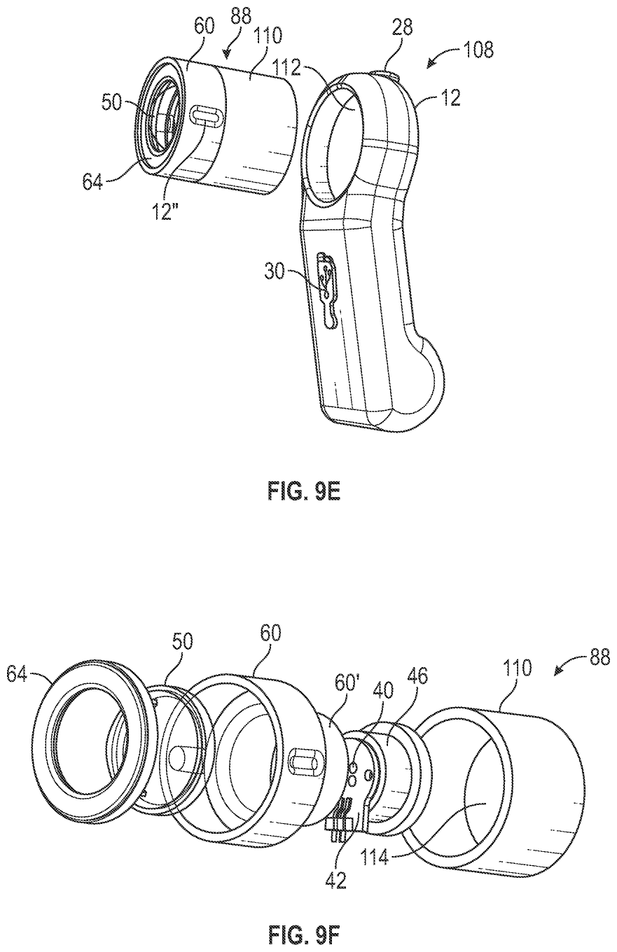

FIG. 9A is a side view of an illumination device 108 that is similar to the illumination device 86 of FIGS. 6A to 6B with a different structure for the illumination cartridge 88 according to further aspects of the present disclosure. FIG. 9B is a front view of the illumination device 108 of FIG. 9A. FIG. 9C is a back perspective view of the illumination device 108 of FIG. 9A. As illustrated, the illumination cartridge 88 is configured to be attached to a frontside of the housing 12. In certain embodiments, the port 30 may be positioned on a portion of the housing 12, such as a handle formed by the housing 12. The illumination device 108 may further comprise the user interface display 106 as described above for FIG. 8C.

FIG. 9D is a cross-sectional view of the illumination device 108 of FIG. 9A with the addition of the light guide 14 and light guide positioner 20. The illumination cartridge 88 comprises the lens 50, the one or more light sources 40, the first board 42, and the heatsink 46 as previously described. In certain embodiments, the illumination cartridge 88 may also comprise the optical cup 60 as described above for FIG. 2D. In certain embodiments, the heatsink 46 may comprise a protrusion 46″ or protruding lip along a backside of the heatsink 46 that is opposite the mounting location of the one or more light sources 40. In this manner, the portion 60′ of the optical cup 60 may abut the protrusion 46″. Stated differently, the heatsink 46 may be inserted into the optical cup 60 such that the protrusion 46″ forms a stopper at the portion 60′ of the optical cup 60. The illumination cartridge 88 may further comprise a base 110 that encloses the heatsink 46 and abuts the optical cup 60. As illustrated, a portion of the base 110 is sized to be attached within the housing 12. In certain embodiments, the illumination cartridge 88 may include the token (32-1 or 32-2) as illustrated in FIG. 6G.

FIG. 9E is front perspective view of the illumination device 108 of FIG. 9A with the illumination cartridge 88 detached from the housing 12. In certain embodiments, the optical cup 60 may form one or more indentations 12″ that are configured to receive the light guide 14. As illustrated, the housing 12 may form a recess 112 along a front face thereof. When fully assembled, the illumination cartridge 88, such as a portion of the base 110, may be inserted within the recess 112. In certain embodiments, outer surfaces of the illumination cartridge 88, such as the optical cup 60 and base 110, may form cylindrical walls that are similar to a form factor of the light guide 14 of FIG. 9D.

FIG. 9F is an exploded view of the illumination cartridge 88 of FIG. 9A. As illustrated, the lens 50 is positioned between the gasket 64 and the optical cup 60. The optical cup 60 is configured to receive the heatsink 46, the one or more light sources 40, and the first board 42 from a side that is opposite the lens 50. Finally, the base 110 may form another recess 114 that is shaped to receive the heatsink 46, the one or more light sources 40, the first board 42, and the portion 60′ of the optical cup 60 that is attached to the heatsink 46.

As previously described, certain embodiments of illumination devices may embody connected devices that are in communication with larger systems that administer, monitor, and/or analyze light treatment protocols across multiple illumination devices in one or more geographic locations. Illumination devices as disclosed herein may be in communication with one or more external devices, such as one or more local devices, networks, and/or servers. Local devices include various local computing devices, such as computers, tablets, and mobile devices, as well as applications on such local devices. Networks may refer to any medium or architecture capable of facilitating communication or data transfer. Examples of the networks include, without limitation, an intranet, a wide area network (WAN), a local area network (LAN), a personal area network (PAN), the Internet, power line communications (PLC), a cellular network (e.g., a global system for mobile communications (GSM) network), or the like. Servers may include server-side applications that collect usage data from illumination devices and/or provide information, such as treatment protocols, back to the illumination devices. Communication between illumination devices as described herein and one or more of the local devices, servers, and networks may involve communication or data transfer using wireless or wired connections.

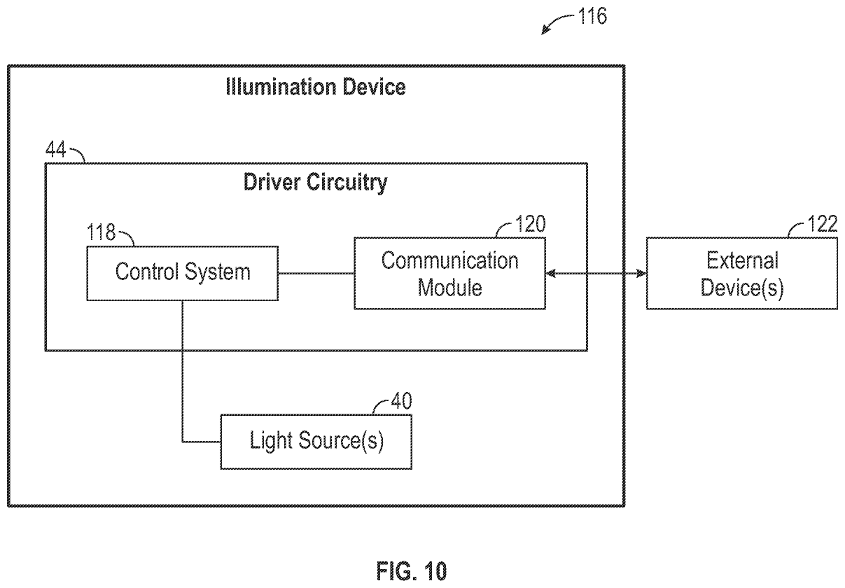

FIG. 10 is a schematic view of an exemplary illumination device 116 configured to communicate with one or more local devices, servers, and networks according to embodiments of the present disclosure. The illumination device 116 is drawn in schematic form and may be a representation of any of the previously described illumination devices, including those illustrated in FIGS. 1A to 1F, FIGS. 2A to 2E, FIGS. 3A to 3B, FIGS. 4A to 4F, FIGS. 5A to 5D, FIGS. 6A to 6G, FIGS. 7A to 7E, FIGS. 8A to 8E, and FIGS. 9A to 9F. In this regard, the driver circuitry 44 as described for FIG. 10 may describe the driver circuitry 44 for any of these previously described embodiments.

As illustrated in FIG. 10, the driver circuitry 44 may include a control system 118 and a communication module 120, among other elements. The communication module 120 may facilitate external communication with one or more external devices 122, such as local devices, servers, and/or networks. The communication module 120 may provide communication via any number of manners, including Bluetooth, wired and/or wireless internet connections, a cellular network, analog communication such as one or more pre-programmed buttons of the illumination device 116, or any other form of analog or digital communication. In certain embodiments, the communication module 120 may be configured to communicate with the token 32 of any of the previously described embodiments. The control system 118 may include light source drivers, among other control circuitry, configured to drive the light sources 40 according to a treatment protocol. In certain embodiments, the control system 118 may further be configured to determine location information associated with the implemented treatment protocol and send the location information to one or more local devices, servers, and/or networks by way of the communication module 120.

It is contemplated that any of the foregoing aspects, and/or various separate aspects and features as described herein, may be combined for additional advantage. Any of the various embodiments as disclosed herein may be combined with one or more other disclosed embodiments unless indicated to the contrary herein.

Those skilled in the art will recognize improvements and modifications to the preferred embodiments of the present disclosure. All such improvements and modifications are considered within the scope of the concepts disclosed herein and the claims that follow.

1. An illumination device comprising:
at least one light source arranged to irradiate light on tissue to induce a biological effect;
driver circuitry configured to drive the at least one light source; and
a removable token configured to control operation of the at least one light source.

2. The illumination device of claim 1, wherein the removable token is configured for near field communication with the driver circuitry.

3. The illumination device of claim 1, further comprising a housing that at least partially encloses the at least one light source and the driver circuitry.

4. The illumination device of claim 3, wherein the housing comprises a receptacle configured to receive the removable token.

5. The illumination device of claim 4, wherein the removable token forms a protrusion with a first shape that corresponds with a second shape of a slot of the receptacle.

6. The illumination device of claim 4, wherein the removable token comprises a plug that fits into the receptacle.

7. The illumination device of claim 4, wherein the removable token forms a physical connection within the receptacle for providing transfer of information.

8. The illumination device of claim 4, wherein the removable token is configured to provide near field communication for transfer of information with the driver circuitry.

9. The illumination device of claim 3, wherein the housing comprises a window proximate the at least one light source configured to provide visual indication when the at least one light source is electrically activated.

10. The illumination device of claim 3, further comprising:
a heatsink within the housing, wherein the at least one light source is on the heatsink; and
an optical cup peripherally surrounding the at least one light source, wherein a portion of the optical cup is attached to the heatsink.

11. The illumination device of claim 3, wherein the housing forms a handle and the removable token is attached to a base of the handle.

12. The illumination device of claim 11, further comprising a carriage within the housing, wherein the removable token is attached to the carriage.

13. The illumination device of claim 12, further comprising a port at the carriage, the port being configured as one or more of a charging port and a data transfer port.

14. The illumination device of claim 12, wherein the driver circuitry is attached to the carriage.

15. The illumination device of claim 4, wherein the removable token is configured to slide within the receptacle to lock in place.

16. The illumination device of claim 3, further comprising:
a light guide attached to the housing and optically coupled to the at least one light source; and
a light guide positioner attached to the light guide, wherein the light guide positioner is configured to be positioned within an oral cavity of a user.

17. The illumination device of claim 3, further comprising a user interface display on the housing.

18. The illumination device of claim 1, wherein the removable token is configured to be prescribed by a health care provider.

19. The illumination device of claim 1, wherein the removable token is configured to control operation of the at least one light source according to a treatment protocol for irradiating light on tissue within a body cavity.

20. The illumination device of claim 1, wherein the removable token forms a switch electrically coupled to the driver circuitry for initiating operation of the at least one light source.

21. The illumination device of claim 1, further comprising a communication module configured to communication with an external device.

22. The illumination device of claim 1, wherein the removable token is part of an illumination cartridge that includes the at least one light source, the illumination cartridge being removable.

23. The illumination device of claim 22, wherein the removable token is removable from the illumination cartridge.

24. The illumination device of claim 1, wherein the removable token is part of an illumination head that includes the at least one light source, the illumination head being removable.

25. The illumination device of claim 24, wherein the removable token is removable from the illumination head.

26-67. (canceled)
